(19) United States
(12) Patent Application Publication
(10) Pub. No.: US 2026/0364066 A1
(43) Pub. Date: Nov. 19, 2026

(54) COMBINATION THERAPIES OF ZELENIRSTAT FOR INHIBITING N-MYRISTOYLTRANSFERASES IN CANCER CELLS

(71) Applicant: PACYLEX PHARMACEUTICALS INC., Calgary (CA)

(72) Inventors: Luc G. BERTHIAUME, Edmonton (CA); Erwan Beauchamp, Edmonton (CA)

(21) Appl. No.: 19/649,305

(22) Filed: Apr. 16, 2026

Related U.S. Application Data
(60) Provisional application No. 63/794,576, filed on Apr. 25, 2025.

(30) Foreign Application Priority Data
Dec. 24, 2025 (CA) 3296980

(51) Int. Cl. A61K 31/496; A61K 31/4184; A61K 31/635; A61K 31/7068; A61P 35/02

(52) U.S. Cl. CPC A61K 31/496; A61K 31/4184; A61K 31/635; A61K 31/7068; A61P 35/02

(57) ABSTRACT

Combination Therapies of Zelenirstat for Inhibiting N-Myristoyltransferases in Cancer Cells.

### FIELD

The present disclosure relates generally to the treatment of cancers with zelenirstat in combination with other chemotherapies.

### BACKGROUND

Myristoylation, the N-terminal modification of proteins with the fatty acid myristate, is critical for membrane targeting of several hundred human proteins [1], including ones critical for intracellular signaling. Because cancer cells often have increased N-myristoyltransferase (NMT) protein expression, NMTs have been proposed as anti-cancer targets.

### SUMMARY

As described herein, there is also provided the following embodiments:
1. A method of treating cancer in a subject comprising: administering a therapeutically effective amount of PCLX-001 and a therapeutically effective amount of cytarabine.
2. The method of embodiment 1, wherein the cancer is bladder cancer, breast cancer, colorectal cancer, leukemia, lung cancer, lymphoma, myeloma, pancreatic cancer or skin cancer.
3. The method of embodiment 1 or embodiment 2, wherein the subject is a human.
4. A method of treating bladder cancer, breast cancer, colorectal cancer, leukemia, lung cancer, lymphoma, myeloma, pancreatic cancer or skin cancer in a subject comprising: administering a therapeutically effective amount of PCLX-001 and a therapeutically effective amount of doxorubicin.
5. The method of embodiment 4, wherein the cancer is bladder cancer, breast cancer, colorectal cancer, leukemia, lung cancer, lymphoma, myeloma, pancreatic cancer or skin cancer.
6. The method of embodiment 4 or embodiment 5, wherein the subject is a human.
7. A method of treating cancer in a subject comprising: administering a therapeutically effective amount of PCLX-001 and a therapeutically effective amount of venetoclax.
8. The method of embodiment 7, wherein the cancer is a hematologic cancer.
9. The method of embodiment 8, wherein the hematologic cancer is myeloma, lymphoma, or leukemia.
10. The method of embodiment 8, wherein the myeloma is plasma cell myeloma.
11. The method of embodiment 8, wherein the lymphoma is B-lymphoblastic lymphoma, T-lymphoblastic lymphoma, anaplastic large-cell lymphoma, Burkitt lymphoma, diffuse large B-cell lymphoma, germinal center B-cell type lymphoma, mantle cell lymphoma, or mature T and natural killer neoplasm.
12. The method of embodiment 8, wherein the leukemia is acute megakaryoblastic leukemia, acute monoblastic/monocytic leukemia, acute myeloid leukemia, B-lymphoblastic leukemia, chronic eosinophilic leukemia, plasma cell leukemia, or T-lymphoblastic leukemia,
13. The method of according to any one of embodiments 7 to 12, wherein the subject is a human.
14. A method of treating cancer in a subject comprising: administering a therapeutically effective amount of PCLX-001 and a therapeutically effective amount of bendamustine.
15. The method of embodiment 14, wherein the cancer is a hematologic cancer.
16. The method of embodiment 15, wherein the hematologic cancer is myeloma, lymphoma, or leukemia.
17. The method of embodiment 15, wherein the myeloma is plasma cell myeloma.
18. The method of embodiment 15, wherein the lymphoma is B-lymphoblastic lymphoma, T-lymphoblastic lymphoma, anaplastic large-cell lymphoma, Burkitt lymphoma, diffuse large B-cell lymphoma, germinal center B-cell type lymphoma, mantle cell lymphoma or mature T and natural killer neoplasm.
19. The method of embodiment 15, wherein the leukemia is acute megakaryoblastic leukemia, acute monoblastic/monocytic leukemia, acute myeloid leukemia, B-lymphoblastic leukemia, chronic eosinophilic leukemia, plasma cell leukemia, or T-lymphoblastic leukemia.
20. The method of according to any one of embodiments 14 to 19, wherein the subject is a human.
21. Use of a therapeutically effective amount of PCLX-001 and a therapeutically effective amount of cytarabine for treating cancer in a subject, or in the manufacture of a medicament for treating cancer in a subject.
22. The use of embodiment 21, wherein the cancer is bladder cancer, breast cancer, colorectal cancer, leukemia, lung cancer, lymphoma, myeloma, pancreatic cancer or skin cancer.
23. The use of embodiment 21 or embodiment 22, wherein the subject is a human.
24. Use of a therapeutically effective amount of PCLX-001 and a therapeutically effective amount of doxorubicin for treating bladder cancer, breast cancer, colorectal cancer, leukemia, lung cancer, lymphoma, myeloma, pancreatic cancer or skin cancer in a subject, or in the manufacture of a medicament for treating bladder cancer, breast cancer, colorectal cancer, leukemia, lung cancer, lymphoma, myeloma, pancreatic cancer or skin cancer in a subject.
25. The use of embodiment 24, wherein the cancer is bladder cancer, breast cancer, colorectal cancer, leukemia, lung cancer, lymphoma, myeloma, pancreatic cancer or skin cancer.
26. The use of embodiment 24 or embodiment 25, wherein the subject is a human.
27. Use of a therapeutically effective amount of PCLX-001 and a therapeutically effective amount of venetoclax for treating cancer in a subject, or in the manufacture of a medicament for treating cancer in a subject.
28. The use of embodiment 27, wherein the cancer is a hematologic cancer.
29. The use of embodiment 28, wherein the hematologic blood cancer is myeloma, lymphoma, or leukemia.
30. The use of embodiment 29, wherein the myeloma is plasma cell myeloma.
31. The use of embodiment 29, wherein the lymphoma is B-lymphoblastic lymphoma, T-lymphoblastic lymphoma, anaplastic large-cell lymphoma, Burkitt lymphoma, diffuse large B-cell lymphoma, germinal center B-cell type lymphoma, mantle cell lymphoma, or mature T and natural killer neoplasm.
32. The use of embodiment 29, wherein the leukemia is acute megakaryoblastic leukemia, acute monoblastic/monocytic leukemia, acute myeloid leukemia, B-lymphoblastic leukemia, chronic eosinophilic leukemia, plasma cell leukemia, or T-lymphoblastic leukemia,
33. The use of according to any one of embodiments 27 to 32, wherein the subject is a human.
34. Use of a therapeutically effective amount of PCLX-001 and a therapeutically effective amount of bendamustine for treating cancer in a subject, or in the manufacture of a medicament for treating cancer in a subject.
35. The use of embodiment 34, wherein the cancer is a hematologic cancer.
36. The use of embodiment 35, wherein the blood cancer is myeloma, lymphoma, or leukemia.
37. The use of embodiment 36, wherein the myeloma is plasma cell myeloma.
38. The use of embodiment 36, wherein the lymphoma is B-lymphoblastic lymphoma, T-lymphoblastic lymphoma, anaplastic large-cell lymphoma, Burkitt lymphoma, diffuse large B-cell lymphoma, germinal center B-cell type lymphoma, mantle cell lymphoma or mature T and natural killer neoplasm.
39. The use of embodiment 36, wherein the leukemia is acute megakaryoblastic leukemia, acute monoblastic/monocytic leukemia, acute myeloid leukemia, B-lymphoblastic leukemia, chronic eosinophilic leukemia, plasma cell leukemia, or T-lymphoblastic leukemia.
40. The use of according to any one of embodiments 34 to 39, wherein the subject is a human.
41. An antibody drug conjugate (ADC) comprising an NMTi and a chemotherapeutic agent conjugated to an antibody, optionally through one or more linkers.
43. The ADC of embodiment 41, wherein the NMTi is PCLX-001 or PCLX-002.
44. An antibody drug conjugate (ADC) comprising PCLX-001 conjugated to an antibody through a linker.
45. An antibody drug conjugate (ADC) comprising PCLX-001 and doxorubicin conjugated to an antibody, optionally through one or more linkers.
46. An antibody drug conjugate (ADC) comprising PCLX-001 and cytarabine conjugated to an antibody, optionally through one or more linkers.
47. An antibody drug conjugate (ADC) comprising PCLX-001 and venetoclax conjugated to an antibody, optionally through one or more linkers.
48. An antibody drug conjugate (ADC) comprising PCLX-001 and bendamustine conjugated to an antibody, optionally through one or more linkers.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Embodiments of the present disclosure will now be described, by way of example only, with reference to the attached Figures.

FIG. 1 depicts empirically derived combination matrices compared to the respective Loewe additivity models for the combination of Cytarabine and UAlberta CCI-002.

FIG. 2 shows a comparison of the EC50 for the panel of 69 cell lines treated with either cytarabine, doxorubicin or zelenirstat.

FIG. 3 shows a distribution of EC50 for the panel of 69 cell lines treated with either cytarabine, doxorubicin or zelenirstat.

FIG. 4 shows cytarabine and zelenirstat combination synergy scores with cell lines sorted by tissue type.

FIG. 5 shows doxorubicin and zelenirstat combination synergy scores with cell lines sorted by tissue type.

FIGS. 6A and 6B illustrate a model depicting proposed PCLX-001 mechanism of action in B cell lymphoma.

FIGS. 7A and 7B depicts results showing dasatinib and ibrutinib do not synergize the cytotoxic effects of PCLX-001 in IM9 and BL2 cells.

FIG. 8 shows a dose response curve representations of cytostatic vs cytotoxic compound activity.

FIG. 9 depicts box whisker plots showing enhance single agent response by tissue type.

FIG. 10 shows a comparison of the EC10, EC50 and EC90 for the panel of 55 hematological cell lines treated with zelenirstat.

FIG. 11 shows a comparison of the EC₅₀ for the panel of 55 hematological cell lines treated with either zelenirstat, venetoclax or bendamustine.

FIG. 12 shows a distribution of EC₅₀ for the panel of 55 hematological cell lines treated with either zelenirstat, venetoclax or bendamustine.

FIG. 13 shows heatmaps of combination activity

FIG. 14 shows experimental results wherein zelenirstat shows moderate synergy with bendamustine.

FIG. 15 shows experimental results wherein zelenirstat shows synergy with venetoclax (ABT-199).

FIG. 16 illustrates a model describing the potential mechanism of action for the strong synergy observed between zelenirstat and venetoclax.

FIG. 17 illustrates Zelenirstat shows synergy with venetoclax. AML cell lines were screened for synergy between zelenirstat and venetoclax using Horizon's Combination Profiling platform. Isobolograms of (A) MV-4-11 and (B) KG-1 cell lines of zelenirstat and venetoclax combination therapy. Trendlines below additivity line indicate synergy. (C) Synergy scores of AML cell lines in combination with venetoclax. Synergy scores indicate growth inhibition in excess of additive effects.

### DETAILED DESCRIPTION

Generally, the present disclosure provides combination therapies of zelenirstat for inhibiting N-myristoyltransferases in Cancer Cells.

PCLX-001 is also known as DDD86418 (WO 2010/026365) and Zelenirstat.

The structure of PCLX-001 is as follows.

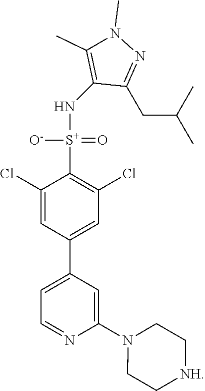 [SMILES: Cc1c(N[S+](=O)([O-])c2c(Cl)cc(-c3ccnc(N4CCNCC4)c3)cc2Cl)c(CC(C)C)nn1C]

The structure of DDD85646 (PCLX-002) is as follows.

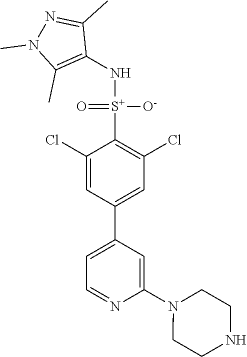 [SMILES: Cc1nn(C)c(C)c1N[S+](=O)([O-])c1c(Cl)cc(-c2ccnc(N3CCNCC3)c2)cc1Cl]

PCLX-001 (also known as DDD86481) and PCLX-002 (also known as DDD85646) are described in WO 2010/026365.

In one example, an N-myristoyltransferase inhibitor (NMTi) is described in WO 2010/026365, the entire contents of which is incorporate by reference.

In some examples, there are provided cancer chemotherapeutic agents. Examples of such chemotherapeutic agents include, but are not limited to, compounds that reduce proliferation of cancer cells and encompass cytotoxic agents and cytostatic agents. Non-limiting examples of chemotherapeutic agents include alkylating, agents, nitrosoureas, antimetabolites, antitumor antibiotics, plant (vinca) alkaloids, and steroid hormones.

Additional examples of chemotherapeutic agents include agents that act to reduce cellular proliferation, antimetabolite agents, natural products and their derivatives, m microtubule affecting agents that have antiproliferative activity

The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. Cancer cells may be in the form of a tumor, but such cells may also exist alone within a subject, or may be a non-tumorigenic cancer cell.

A cancer may be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

It will be appreciated that, in general, determination of the severity of disease requires identification of certain disease characteristics, for example, whether the cancer is pre-metastatic or metastatic, the stage and/or grade of cancer, and the like.

Staging is a process used to describe how advanced a cancer is in a subject. Staging may be important in determining a prognosis, planning treatment and evaluating the results of such treatment. While different cancer staging systems may need to be used for different types of cancer, most staging systems generally involve describing how far the cancer has spread anatomically and attempt to put subjects with similar prognosis and treatment in the same staging group.

Examples of common staging systems used for most solid tumours, some leukemias and lymphomas are the Overall Stage Grouping system and the TMN system. In the Overall Stage Grouping system, Roman numerals I through IV are utilized to denote the four stages of a cancer. Generally, if a cancer is only detectable in the area of the primary lesion without having spread to any lymph nodes it is called Stage I. Stage II and III cancers are generally locally advanced and/or have spread to the local lymph nodes. For example, if the cancer is locally advanced and has spread only to the closest lymph nodes, it is called Stage II. In Stage III, the cancer is locally advanced and has generally spread to the lymph nodes in near proximity to the site of the primary lesion. Cancers that have metastasized from the primary tumour to a distant part of the body, such as the liver, bone, brain or another site, are called, Stage IV, the most advanced stage. Accordingly, stage I cancers are generally small localized cancers that are curable, while stage IV cancers usually represent inoperable or metastatic cancers. As with other staging systems, the prognosis for a given stage and treatment often depends on the type of cancer. For some cancers, classification into four prognostic groups is insufficient and the overall staging is further divided into subgroups. In contrast, some cancers may have fewer than four stage groupings.

A cancer that recurs after all visible tumour has been eradicated is called recurrent disease, with local recurrence occurring in the location of the primary tumour and distant recurrence representing distant metastasis.

Variations to the staging systems may depend on the type of cancer. Moreover, certain types of cancers. The staging system for individual cancers may be revised with new information and subsequently, the resulting stage may change the prognosis and treatment for a specific cancer.

The “grade” of a cancer may be used to describe how closely a tumour resembles normal tissue of its same type. Based on the microscopic appearance of a tumour, pathologists identify the grade of a tumour based on parameters such as cell morphology, cellular organization, and other markers of differentiation. As a general rule, the grade of a tumour corresponds to its rate of growth or aggressiveness and tumours are typically classified from the least aggressive (Grade I) to the most aggressive (Grade IV).

Accordingly, the higher the grade, the more aggressive and faster growing the cancer. Information about tumour grade is useful in planning treatment and predicting prognosis.

In some examples, in the case of lymphoma, Stage 1 refers to lymphoma in only one group of lymph nodes. Stage II refers to two or more groups of lymph nodes are affected but they are all either above or below the diaphragm, either all in the chest or all in the abdomen. Stage III refers to two or more groups of lymph nodes are affected in both the chest and the abdomen. Stage IV refers to lymphoma is in at least one organ (e.g., bone marrow, liver or lungs) as well as the lymph nodes. Additional designations may be added to the foregoing stages. For example, “A” generally means the patient has not experiences any troublesome symptoms. “B” means the patient has experienced B symptoms (e.g., fever, night sweats, weight loss). X means the patient has bulky disease (e.g., large tumour greater than 10 cm in size). E means the patient has extranodal disease (e.g., disease outside the lymph nodes).

In a specific example, the cancer is a lymphoma.

The term “lymphoma” generally refers to a malignant neoplasm of the lymphatic system, including cancer of the lymphatic system. The two main types of lymphoma are Hodgkin's disease (HD or HL) and non-Hodgkin's lymphoma (NHL). Abnormal cells appear as congregations which enlarge the lymph nodes, form solid tumours in the body, or more rarely, like leukemia, circulate in the blood. Hodgkin's disease lymphomas, include nodular lymphocyte predominance Hodgkin's lymphoma; classical Hodgkin's lymphoma; nodular sclerosis Hodgkin's lymphoma; lymphocyterich classical Hodgkin's lymphoma; mixed cellularity Hodgkin's lymphoma; lymphocyte depletion Hodgkin's lymphoma. Non-Hodgkin's lymphomas include small lymphocytic NHL, follicular NHL; mantle cell NHL; mucosa-associated lymphoid tissue (MALT) NHL; diffuse large cell B-cell NHL; mediastinal large B-cell NHL; precursor T lymphoblastic NHL; cutaneous T-cell NHL; T-cell and natural killer cell NHL; mature (peripheral) T-cell NHL; Burkitt's lymphoma; mycosis fungoides; Sezary Syndrome; precursor B-lymphoblastic lymphoma; B-cell small lymphocytic lymphoma; lymphoplasmacytic lymphoma; splenic marginal zone B-cell lymphoma; nodal marginal zone lymphoma; plasma cell myeloma/plasmacytoma; intravascular large B-cell NHL; primary effusion lymphoma; blastic natural killer cell lymphoma; enteropathy-type T-cell lymphoma; hepatosplenic gamma-delta T-cell lymphoma; subcutaneous panniculitis-like T-cell lymphoma; angioimmunoblastic Tcell lymphoma; primary systemic anaplastic large T/null cell lymphoma, diffuse large B-cell lymphoma (DLBCL), refractory diffuse large B-cell lymphoma (DLBCL).

In a specific example, the lymphoma is a B-cell lymphoma.

In one example, the cancer is diffuse large B-cell lymphoma (DLBCL).

In one example, the cancer is refractory diffuse large B-cell lymphoma (DLBCL).

In some examples, the compositions and/or compositions described herein (for example, PCLX-001) may be used to treat various stages and grades of cancer development and progression. In some examples, PCLX-001 may be used in the treatment of early stage cancers including early neoplasias that may be small, slow growing, localized and/or nonaggressive, for example, with the intent of curing the disease or causing regression of the cancer, as well as in the treatment of intermediate stage and in the treatment of late stage cancers including advanced and/or metastatic and/or aggressive neoplasias, for example, to slow the progression of the disease, to reduce metastasis or to increase the survival of the patient. Similarly, PCLX-001 may be used in the treatment of low grade cancers, intermediate grade cancers and or high grade cancers.

In some examples, it is contemplated that PCLX-001 may be used in the treatment of indolent cancers, recurrent cancers including locally recurrent, distantly recurrent and/or refractory cancers (i.e., cancers that have not responded to treatment), metastatic cancers, locally advanced cancers and aggressive cancers.

In another example, the cancer is leukemia, myeloma, breast cancer, small cell lung cancer, non-small cell lung cancer cells; melanoma, adenocarcinoma, pancreas cancer, bladder cancer, ovarian cancer, brain cancer, colon cancer, neuroblastoma, carcinoma, endometrial cancer, sarcoma, thyroid cancer, fibrosarcoma, oral cancer, tongue cancer, nasopharynx cancer, larynx cancer, esophageal cancer, germ cell cancer, stomach cancer, hepatocellular cancer, biliary tract cancer, small intestinal cancer, testis cancer, epidermoid cancer, cervical cancer, kidney cancer, prostate cancer, or pharynx cancer

In some examples, PCLX-001 may be used in combination with one or more therapeutic agents as part of a primary therapy or an adjuvant therapy. “Primary therapy” or “first-line therapy” refers to treatment upon the initial diagnosis of cancer in a subject. Exemplary primary therapies may involve surgery, a wide range of chemotherapies, immunotherapy and/or radiotherapy. When first-line or primary therapy is not systemic chemotherapy or immunotherapy, then subsequent chemotherapy or immunotherapy may be considered as “first-line systemic therapy”. In one example, PCLX-001 may be used for first-line systemic therapy.

The term “adjuvant therapy” refers to a therapy that follows a primary therapy and that is administered to subjects at risk of relapsing. Adjuvant systemic therapy is typically begun soon after primary therapy to delay recurrence, prolong survival or cure a subject. Treatment of a refractory cancer may be termed a “second-line therapy” and is a contemplated use of the present invention, in addition to first-line therapy.

In one embodiment of the present invention, the combinations are used in the treatment of an early stage cancer. In another embodiment, the combinations are used as a first-line systemic therapy for an early stage cancer.

In an alternate example, PCLX-001 may be used in the treatment of a late stage and/or advanced and/or metastatic cancer. In a further embodiment, PCLX-001 may be administered as a first-line systemic therapy for the treatment of a late stage and/or advanced and/or metastatic cancer.

A refractory cancer refers to a cancer that is not amenable to surgical intervention and the cancer is either initially unresponsive to chemo- or radiation therapy or the cancer becomes unresponsive over time.

The term “relapse” refers to those patients who achieved a clinical response (CR) or partial response (PR) and subsequently experienced disease progression.

The term “clinical response” refers to inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others.

The term “overall survival” or “OS” refers to the time from enrollment to death from any cause.

The term “duration of response” or “DOR” refers to the time from the first occurrence of a documented objective response to disease progression.

The term “progression free survival” or “PFS” refers to the time from enrollment to the date of the first recorded occurrence of disease progression.

The term “stable disease” or “SD” refers to neither sufficient shrinkage to qualify for CR or PR nor sufficient increase growth of tumor to qualify for PD.

The term “partial response” refers to persistence of one or more non-target lesions and/or (if applicable) maintenance of tumor marker level above the normal limits.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal.

In a specific example, the subject is a human.

The term “treatment” or “treat” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early cancer, for example an early stage lymphoma, can be treated to prevent progression or alternatively a subject in remission can be treated with a compound or composition described herein to prevent recurrence.

The term “pharmaceutically acceptable” as used herein includes compounds, materials, compositions, and/or dosage forms (such as unit dosages) which are suitable for use in contact with the tissues of a subject without excessive toxicity, irritation, allergic response, or other problem or complication, commensurate with a reasonable benefit/risk ratio. Each carrier, excipient, etc. is also be “acceptable” in the sense of being compatible with the other ingredients of the formulation.

The term “excipient” means a pharmacologically inactive component such as a diluent, lubricant, surfactant, carrier, or the like. Excipients that are useful in preparing a pharmaceutical composition are generally safe, non-toxic and are acceptable for human pharmaceutical use. Reference to an excipient includes both one and more than one such excipient.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), stabilizers and preservatives, and the like.

A “treatment or dosage regimen” as used herein refers to a combination of dosage, frequency of administration, or duration of treatment, with or without addition of a second medication.

The term “diagnosis” as used herein, refers to the identification of a molecular and/or pathological state, disease or condition, such as the identification of lymphoma, or other type of cancer.

The term “alleviates” as used herein refers to a decrease, reduction or elimination of a condition, disease, disorder, or phenotype, including an abnormality or symptom.

In some aspects there is provided a pharmaceutical composition comprising PCLX-001 in admixture with a pharmaceutically acceptable adjuvant, diluent or carrier.

Pharmaceutical compositions of the present disclosure may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example.

Solid dosage forms for oral administration include capsules, tablets, pills, powders and granules. In such solid dosage forms, an active compound (e.g., PCLX-001) is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

The term “solid” as used herein refers to a substance that is solid or semi-solid at room temperature. Hence, as used herein, a “solid” substance may become liquid at, e.g., body temperature.

The term “Dosage form” refers to a form of a formulation that contains an amount of active agent or prodrug of an active agent, e.g., PCLX-001, which may be administered to a patient to achieve a therapeutic effect.

An oral dosage form is intended to be administered to a patient taken orally, for example via the mouth and swallowed.

The terms “taken orally” and “oral administration” refer to a manner of providing an active agent to a subject or patient by the mouth through the gastrointestinal tract (digestive tract, digestional tract, GI tract, GIT, gut, or alimentary canal) and are used interchangeably. The gastrointestinal tract is an organ system within humans and other animals which takes in food, digests it to extract and absorb energy and nutrients, and expels the remaining waste as feces. The mouth, esophagus, stomach and intestines are part of the gastrointestinal tract.

Suitably, oral formulations may contain a dissolution aid. The dissolution aid is not limited as to its identity so long as it is pharmaceutically acceptable. Examples include non-ionic surface active agents, such as sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylamides, and alkylamine oxides; bile acid and salts thereof (e.g. chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

PCLX-001 may also be in a micro-encapsulated form, if appropriate, with one or more of the above-mentioned excipients.

Liquid dosage forms for oral administration include pharmaceutically acceptable emulsions, solutions, suspensions, syrups and elixirs. In addition to PCLX-001, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

In other examples, pharmaceutical compositions of the present disclosure may be used for parenteral injection suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

Prolonged absorption of an injectable pharmaceutical form may be brought about by the inclusion of agents (for example aluminum monostearate and gelatin) which delay absorption.

In some cases, in order to prolong the effect of the pharmaceutical compositions of the present disclosure, it is desirable to slow the absorption of the composition, or PCLX-001 from subcutaneous or intramuscular injection. This may be accomplished by the use of a liquid suspension, where PCLX-001 is suspended in. The rate of absorption of PCLX-001 then depends upon its rate of dissolution.

Alternatively, delayed absorption of a parenterally administered pharmaceutical composition of the present disclosure is accomplished by dissolving or suspending PCLX-001 in an oil vehicle. Injectable depot forms are suitably made by forming microencapsulated matrices of PCLX-001 in biodegradable polymers, for example polylactide-polyglycolide. Depending upon the ratio of PCLX-001 to polymer and the nature of the particular polymer employed, the rate of release can be controlled. Examples of other biodegradable polymers include poly(orthoesters) and poly(anhydrides).

Depot injectable formulations may also be prepared by entrapping PCLX-001 in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

In other examples, pharmaceutical compositions of the present disclosure for rectal or vaginal administration are preferably suppositories which can be prepared by mixing PCLX-001 with suitable non-irritating excipients or carriers such as cocoa butter, polyethylene glycol or a suppository wax which are solid at room temperature but liquid at body temperature and therefore melt in the rectum or vaginal cavity and release PCLX-001.

PCLX-001 may also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. Pharmaceutical compositions of the present disclosure in liposome form can contain, in addition to PCLX-001, stabilisers, preservatives, excipients and the like. The preferred lipids are the phospholipids and the phosphatidyl cholines (lecithins), both natural and synthetic. Methods to form liposomes are known in the art.

Dosage forms for topical administration of PCLX-001 include powders, sprays, ointments and inhalants. PCLX-001 may be mixed under sterile conditions with a pharmaceutically acceptable carrier and any needed preservatives, buffers or propellants which may be required. PCLX-001 may also be administered, for example as part of a pharmaceutical composition, in ophthalmic formulations, or eye ointments, powders and solutions.

In some examples, there is provided antibody-drug conjugates comprising or consisting of PCLX-001 in the treatment of cancer.

Antibody-drug conjugates (ADCs) are a growing drug classes in oncology therapy. ADCs are generally composed of an antibody, such as monoclonal antibodies (mAbs), linked to cytotoxic agents and are designed to improve the therapeutic efficacy of cytotoxic agents by targeted delivery to cells that express the specific target molecules, such as target antigen of interest.

Antibody-drug conjugates (ADCs) may selectively deliver cytotoxic small molecules to targeted cancer cells, leading to an enhanced therapeutic index through increased antitumor activity (tumor cytotoxicity).

The term “antibody-drug conjugate” or “ADC” refers to a conjugate comprising an antibody conjugated to one or more cytotoxic agents, optionally through one or more linkers.

The term “cytotoxic agent” as used herein, refers to a substance that inhibits or prevents a cellular function and/or causes cell death or destruction. The cytotoxic agent can be an anti-angiogenic agent, a pro-apoptotic agent, an anti-mitotic agent, an anti-kinase agent, an alkylating agent, a hormone, a hormone agonist, a hormone antagonist, a chemokine, a drug, a prodrug, a toxin, an enzyme, an antimetabolite, an antibiotic, an alkaloid, or a radioactive isotope. In some examples, a cytotoxic agent is PCLX-001, cytarabine, doxorubicin, venetoclax, or bendamustine.

In some examples, a cytotoxic agent may be referred to as a payload.

In one example, there is provided an antibody drug conjugate (ADC) comprising PCLX-001 conjugated to an antibody via a linker, or a salt thereof.

In one example, there is provided an antibody drug conjugate (ADC) comprising PCLX-001 and doxorubicin conjugated to an antibody optionally through one or more linkers.

In one example, there is provided an antibody drug conjugate (ADC) comprising PCLX-001 and cytarabine conjugated to an antibody optionally through one or more linkers.

In one example, there is provided an antibody drug conjugate (ADC) comprising PCLX-001 and venetoclax conjugated to an antibody optionally through one or more linkers.

In one example, there is provided an antibody drug conjugate (ADC) comprising PCLX-001 and bendamustine conjugated to an antibody optionally through one or more linkers.

A “linker”, as used herein, refers to a molecule that connects one composition to another, e.g., an antibody to an agent. In a specific example, a linker may conjugate an antibody to a cytotoxic agent.

In some examples, a linker may include a labile linker, an acid labile linker, a photolabile linker, a charged linker, a disulfide-containing linker, a peptidase-sensitive linker, a glucuronide-linker, a dimethyl linker, a thio-ether linker, and a hydrophilic linker.

A linker can be cleavable or non-cleavable.

An “antibody” includes, but is not limited to, certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Example 1

In this example, the cytotoxic activity of 2 combinations (Cytarabine & Zelenirstat and Doxorubicin & Zelenirstat) across a panel of 69 cancer cell lines was tested utilising Horizon Discovery's High Throughput Screening platform. Combinations were dosed in a 9×9 format. Single agents were dosed with 8 dose points including the no treatment control resulting in 64 dose ratio combination points. Principal assay readout was growth inhibition determined using a 96-hour ATPlite viability assay. Results have been supplied as metrics tables, Chalice Analyzer files and raw luminescence data files.

Cytarabine (Ara-C) is an antimetabolite chemotherapy drug that may be used for treating acute myeloid leukemia (AML), acute lymphoblastic leukemia (ALL), and certain lymphomas. It may act as a pyrimidine analog, entering cells via nucleoside transporters and converting into its active form, cytarabine triphosphate (Ara-CTP). Ara-CTP is incorporated into DNA during S-phase, halting replication by inhibiting DNA polymerase a and p, leading to DNA strand breaks and apoptosis. Its half-life requires continuous infusion or frequent dosing, and it is often combined with anthracyclines (e.g., daunorubicin) in AML therapy. Cytarabine may be effective in central nervous system (CNS) malignancies due to its ability to cross the blood-brain barrier at high doses.

Doxorubicin is an anthracycline chemotherapy drug that may be used to treat various cancers, including breast cancer, lymphomas, leukemias, and sarcomas. Its mechanism of action involves multiple pathways, for example: (1) Intercalation into DNA, where it inserts between base pairs, disrupting DNA replication and transcription; (2) Inhibition of topoisomerase II, preventing DNA unwinding and causing double-strand breaks, leading to apoptosis; and (3) Generation of reactive oxygen species (ROS), which induces oxidative damage to cellular components. Doxorubicin is highly effective but may be associated with dose-dependent cardiotoxicity, limiting its long-term use. It may be combined with other agents, such as cyclophosphamide or paclitaxel, to enhance efficacy in various cancers.

### Example 1: Zelenirstat Combination with Cytarabine or Doxorubicin

### Method

### Anti-Proliferation Assay

The method by which Horizon performed the High Throughput Screen is described below. The endpoint readout of this assay is based upon quantitation of ATP as an indicator of viable cells (except when noted in Analyzer).

Cell lines that have been preserved in liquid nitrogen are thawed and expanded in growth media. Once cells have reached expected doubling times, screening begins. Cells are seeded in growth media in black 384-well tissue culture treated plates at 500 cells per well (except where noted in Analyzer). Cells are equilibrated in assay plates via centrifugation and placed in incubators (attached to the Dosing Modules) at 37 °C for twenty-four hours before treatment. At the time of treatment, a set of assay plates (which do not receive treatment) are collected and ATP levels are measured by adding ATPLite (Perkin Elmer). These Tzero (To) plates are read using ultra-sensitive luminescence on Envision plate readers. Assay plates are incubated with compound for ninety-six hours (except where noted in Analyzer) and are then analyzed using ATPLite. All data points are collected via automated processes and are subject to quality control and analyzed using Horizon's proprietary software. Assay plates are accepted if they pass the following quality control standards: relative raw values are consistent throughout the entire experiment, Z-factor scores are greater than 0.6 and untreated/vehicle controls behave consistently on the plate.

Horizon utilizes Growth Inhibition (GI) as a measure of cell growth. The GI percentages are calculated by applying the following test and equation:

$$If T < {V}_{0} : 100 * ( 1 - \frac{T - {V}_{0}}{{V}_{0}} )$$

$$If T \geq {V}_{0} : 100 * ( 1 - \frac{T - {V}_{0}}{V - {V}_{0}} )$$

where T is the signal measure for a test article, V is the untreated/vehicle-treated control measure, and V₀ is the untreated/vehicle control measure at time zero (also colloquially referred as T₀ plates). This formula is derived from the Growth Inhibition calculation used in the National Cancer Institute's NCI-60 high throughput screen. For the purposes of this example, all data analysis was performed in Growth Inhibition (except where noted).

A GI reading of 0% represents no growth inhibition and would occur in instances where the T reading at ninety-six hours is comparable to the V reading at the respective time period. A GI 100% represents complete growth inhibition (cytostasis) and in this case cells treated with compound for ninety-six hours would have the same endpoint reading as T₀ control cells. A GI of 200% represents complete death (cytotoxicity) of all cells in the culture well and in this case the T reading at seventy-two hours will be lower than the T₀ control (values near or at zero).

Horizon also provides Inhibition as a measure of cell viability. Inhibition levels of 0% represent no inhibition of cell growth by treatment. Inhibition of 100% represents no doubling of cell numbers during the treatment window. Both cytostatic and cytotoxic treatments can yield an Inhibition percentage of 100%. Inhibition percentage is calculated as the following:

I=1−T/U
where T is the treated and U is the untreated/vehicle control.

### Combination Effect Reference Models

Combination effects may be characterized by comparing each data point to that of a combination reference model that was derived from the single agent curves. Three models are generally used: (1) the Highest Single Agent (HSA) is a reference model where the expected combination effect is the maximum of the single agent responses at corresponding concentrations; (2) the Bliss independence model represents the statistical expectation for independent competing inhibitors; and (3) the Loewe additivity model represents the expected response if both agents are actually the same compound and is the most generally accepted reference for synergy. Both HSA and Bliss are calculated. Determining Loewe additivity requires single agent dose curves, data interpolation and iterative root finding for computation of the Loewe additive response surface.

### Highest Single Agent Model

The HSA model is based simply on the intuition that if a combination's effect exceeds the effect level of each of its constituents, there must be some combination interaction. This model is generally selected for the analysis of combinations where there is a limited dose range or ratio available. Mathematically, the HSA model describes simple superposition of the single and curves:

I_{HSA}(C_{X},C_{Y})=max(I_{X},I_{Y})

where C_{X,Y} are the concentrations of the X and Y compound, and I_{X} and I_{Y} are inhibitions of the single agents at C_{X,Y}. It is also useful to calculate a volume score (HSA Volume) between the data and the HSA surface to characterize the overall strength of combination effects.

### Bliss Independence Model

The Bliss independence model corresponds to multiplicative probabilities for effects in growth measures, and is the preferred reference for synergy in some contexts, especially genetics. Bliss boosting, which models boosts in efficacy at high concentrations different from what the single agents can achieve, is adapted from the Bliss independence model that corresponds to a multiplicative effect in growth measures. The Bliss calculation is:

I_{Bliss}(C_{X},C_{Y})=I_{X}+I_{Y}−I_{X}I_{Y}
where C_{X,Y} are the concentrations of the X and Y compound, and I_{X} and I_{Y} are inhibitions of the single agents at C_{X,Y}. The Bliss volume score represents the statistical expectation for independent competing inhibitors. Bliss independence may be directly calculated from minimally sampled experiments without single agent response curve interpolation or iterative root finding.

### Loewe Additivity Model

The Loewe additivity model is dose-based and applies only to the activity levels achieved by the single agents. Loewe Volume is used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for Loewe additivity is:

I_{Loewe} that satisfies(X/X_{I})+(Y/Y_{I})=1

where X_{I} and Y_{I} are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

Activity observed in excess of Loewe additivity may identify potential synergistic interaction. For the present analysis, as seen in the example below for the combination of Cytarabine and UAlberta CCI-002, empirically derived combination matrices were compared to their respective Loewe additivity models constructed from experimentally collected single agent dose response curves. Summation of this excess additivity across the dose response matrix is referred to as Loewe Volume. Positive Loewe Volume suggests potential synergy, while negative Loewe Volume suggests potential antagonism.

### Synergy Score Analysis

To measure combination effects in excess of Loewe additivity, Horizon has devised a scalar measure to characterize the strength of synergistic interaction termed the Synergy Score. The Synergy score is calculated as:

Synergy Score=log f_{X} log f_{Y}Σmax(0,I_{data})(I_{data}−I_{Loewe})

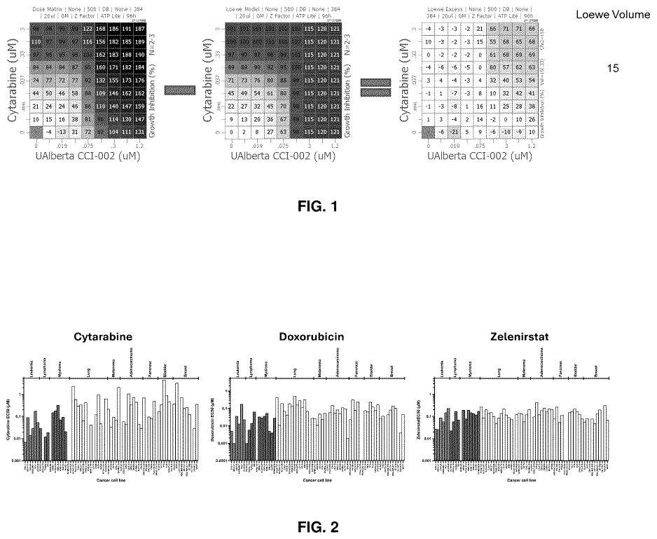

FIG. 1 depicts empirically derived combination matrices compared to the respective Loewe additivity models for the combination of Cytarabine and UAlberta CCI-002.

The fractional inhibition for each component agent and combination point in the matrix is calculated relative to the median of all untreated/vehicle-treated control wells. The Synergy Score equation integrates the experimentally-observed activity volume at each point in the matrix in excess of a model surface numerically derived from the activity of the component agents using the Loewe model for additivity. Additional terms in the Synergy Score equation (above) may be used to normalize for various dilution factors used for individual agents and to allow for comparison of synergy scores across an entire experiment. The inclusion of positive inhibition gating or an I_{data} multiplier removes noise near the zero-effect level, and biases results for synergistic interactions at that occur at high activity levels. Combinations with higher maximum Growth Inhibition (GI) effects or those which are synergistic at low concentrations will have higher Synergy Scores.

Potency shifting was evaluated using an isobologram, which demonstrates how much less drug is required in combination to achieve a desired effect level, when compared to the single agent doses needed to reach that effect. The isobologram was drawn by identifying the locus of concentrations that correspond to crossing the indicated inhibition level. This is done by finding the crossing point for each single agent concentration in a dose matrix across the concentrations of the other single agent. Practically, each vertical concentration C_{Y} is held fixed while a bisection algorithm is used to identify the horizontal concentration C_{X} in combination with that vertical dose that gives the chosen effect level in the response surface Z(C_{X}, C_{Y}). These concentrations are then connected by linear interpolation to generate the isobologram display. For synergistic interactions, the isobologram contour fall below the additivity threshold and approaches the origin, and an antagonistic interaction would lie above the additivity threshold. The error bars represent the uncertainty arising from the individual data points used to generate the isobologram. The uncertainty for each crossing point is estimated from the response errors using bisection to find the concentrations where Z−σ_{Z}(C_{X}, C_{Y}) and Z+σ_{Z}(C_{X}, C_{Y}) cross I_{cut}, where σ_{Z} is the standard deviation of the residual error on the effect scale.

### Combination Index

Potency shifting can also be scored using combination index (CI). For a chosen iso-effect level (I_{Cut}), CI is calculated as:

CI=(C_{X}/EC_{X})+(C_{Y}/EC_{Y})
where (C_{X}/EC_{X}) for a particular data point is the ratio of the X compound's measured concentration to its effective concentration at the chosen effect level. The CI may be an estimate of how much drug was needed in combination relative to the single agent doses required to achieve the chosen effect level. CI values in the range of about 0.5 to about 0.7 are typical for in vitro measurements of current clinical combinations. The CI error (σCI) is calculated using standard error propagation through the CI calculation based on the isobologram errors. In the Chalice Analyzer, the Best CI is reported from the many CI values calculated for each I_{cut} crossing combination. Among all the measured CI values, the one with the largest single-to-noise level (1-CI)/σCI is reported as the Best CI. Best CI Level is the effect level from which the CI is calculated.

### Results

TABLE 1
Cell line panel for synergy between PCLX-001 (zelenirstat or CCI-002)
and Doxorubicine or Cytarabine.

| | | [symbol] | | | |
|---|---|---|---|---|---|
| [symbol] | [symbol] | Type | [symbol] | [symbol] | [symbol] |
| 1 | | | | | |
| 2 | | | | | |
| 3 | | | | | |
| 4 | | Lymphoma | | | |
| 5 | | Lymphoma | | | |
| 6 | | Lung | | | |
| 7 | | Breast | | | |
| 8 | | Lung | | | |
| 9 | | Breast | | | |
| 10 | | | | | |
| 11 | | | | | |
| 12 | | Lung | | | |
| 13 | | | | | |
| 14 | | | | | |
| 15 | | | | | |
| 16 | | Lung | | | |
| 17 | | Lymphoma | | | |
| 18 | | Lung | | | |
| 19 | | Lymphoma | | | |
| 20 | | Breast | | | |
| 21 | | | | | |
| 22 | | Lung | | | |
| 23 | | Lung | | | |
| 24 | | Breast | | | |
| 25 | | | | | |
| 26 | | Lymphoma | | | |
| 27 | | | | | |
| 28 | | | | | |
| 29 | | | | | |
| 30 | | | | | |
| 31 | | Lymphoma | | | |
| 32 | | Lymphoma | | | |
| 33 | | Lung | | | |
| 34 | | Lung | | | |
| 35 | | | | | |
| 36 | | Breast | | | |
| 37 | | Breast | | | |
| 38 | | Breast | | | |
| 39 | | | | | |
| 40 | | Lymphoma | | | |
| 41 | | Lung | | | |
| 42 | | | | | |
| 43 | | | | | |
| 44 | | Lung | | | |
| 45 | | Lung | | | |
| 46 | | Lung | | | |
| 47 | | Lung | | | |
| 48 | | Lymphoma | | | |
| 49 | | Lymphoma | | | |
| 50 | | | | | |
| 51 | | | | | |
| 52 | | | | | |
| 53 | | | | | |
| 54 | | Lymphoma | | | |
| 55 | | | | | |
| 56 | | Bladder | | | |
| 57 | | | | | |
| 58 | | | | | |
| 59 | | Lung | | | |
| 60 | | | | | |
| 61 | | | | | |
| 62 | | | | | |
| 63 | | Bladder | | | |
| 64 | | | | | |
| 65 | | Breast | | | |
| 66 | | Lymphoma | | | |
| 67 | | Bladder | | | |
| 68 | | Breast | | | |
| 69 | | Breast | | | |

[symbol]

indicates data missing or illegible when filed

TABLE 2
Cytarabine Single agent response profile. Table describing
IC50, EC50, GI50 and Max response for the panel of 69
cell lines treated with cytarabine for 96 hours.

| | Cell | | | | | Max | Number |
|---|---|---|---|---|---|---|---|
| | Line | Compound | IC50 | EC50 | GI50 | Response | of Rep- |
| Count | Name | Name | [symbol] | [symbol] | [symbol] | [symbol] | licates |
| 1 | [symbol] | Cytarabine | [symbol] | [symbol] | [symbol] | [symbol] | [symbol] |
| 2 | [symbol] | Cytarabine | [symbol] | [symbol] | [symbol] | [symbol] | [symbol] |
| 3 | [symbol] | Cytarabine | [symbol] | [symbol] | [symbol] | [symbol] | [symbol] |
| 4 | [symbol] | Cytarabine | [symbol] | [symbol] | [symbol] | [symbol] | [symbol] |
| 5 | [symbol] | Cytarabine | [symbol] | [symbol] | [symbol] | [symbol] | [symbol] |
| 6 | [symbol] | Cytarabine | [symbol] | [symbol] | [symbol] | [symbol] | [symbol] |
| 7 | [symbol] | Cytarabine | [symbol] | [symbol] | [symbol] | [symbol] | [symbol] |
| 8 | [symbol] | Cytarabine | [symbol] | [symbol] | [symbol] | [symbol] | [symbol] |
| 9 | [symbol] | Cytarabine | [symbol] | [symbol] | [symbol] | [symbol] | [symbol] |
| 10 | [symbol] | Cytarabine | [symbol] | [symbol] | [symbol] | [symbol] | [symbol] |
| 11 | [symbol] | Cytarabine | [symbol] | [symbol] | [symbol] | [symbol] | [symbol] |
| 12 | [symbol] | Cytarabine | [symbol] | [symbol] | [symbol] | [symbol] | [symbol] |
| 13 | [symbol] | Cytarabine | N/A | N/A | N/A | N/A | [symbol] |
| 14 | [symbol] | Cytarabine | [symbol] | [symbol] | [symbol] | [symbol] | [symbol] |
| 15 | [symbol] | Cytarabine | [symbol] | [symbol] | [symbol] | [symbol] | [symbol] |
| 16 | [symbol] | Cytarabine | [symbol] | [symbol] | [symbol] | [symbol] | [symbol] |
| 17 | [symbol] | Cytarabine | [symbol] | [symbol] | [symbol] | [symbol] | [symbol] |
| 18 | [symbol] | Cytarabine | [symbol] | [symbol] | [symbol] | [symbol] | [symbol] |
| 19 | [symbol] | Cytarabine | [symbol] | [symbol] | [symbol] | [symbol] | [symbol] |
| 20 | [symbol] | Cytarabine | [symbol] | [symbol] | [symbol] | [symbol] | [symbol] |
| 21 | [symbol] | Cytarabine | [symbol] | [symbol] | [symbol] | [symbol] | [symbol] |
| 22 | [symbol] | Cytarabine | [symbol] | [symbol] | [symbol] | [symbol] | [symbol] |
| 23 | [symbol] | Cytarabine | [symbol] | [symbol] | [symbol] | [symbol] | [symbol] |
| 24 | [symbol] | Cytarabine | N/A | N/A | N/A | N/A | [symbol] |
| 25 | [symbol] | Cytarabine | | [symbol] | | [symbol] | [symbol] |
| 26 | [symbol] | Cytarabine | [symbol] | [symbol] | [symbol] | [symbol] | [symbol] |
| 27 | [symbol] | Cytarabine | [symbol] | [symbol] | [symbol] | [symbol] | [symbol] |
| 28 | [symbol] | Cytarabine | [symbol] | [symbol] | [symbol] | [symbol] | [symbol] |
| 29 | [symbol] | Cytarabine | N/A | N/A | N/A | N/A | [symbol] |
| 30 | [symbol] | Cytarabine | [symbol] | [symbol] | [symbol] | [symbol] | [symbol] |
| 31 | [symbol] | Cytarabine | [symbol] | [symbol] | [symbol] | [symbol] | [symbol] |
| 32 | [symbol] | Cytarabine | [symbol] | [symbol] | [symbol] | [symbol] | [symbol] |
| 33 | [symbol] | Cytarabine | | [symbol] | [symbol] | [symbol] | [symbol] |
| 34 | [symbol] | Cytarabine | | [symbol] | [symbol] | [symbol] | [symbol] |
| 35 | [symbol] | Cytarabine | [symbol] | [symbol] | [symbol] | [symbol] | [symbol] |
| 36 | [symbol] | Cytarabine | [symbol] | [symbol] | [symbol] | [symbol] | [symbol] |
| 37 | [symbol] | Cytarabine | [symbol] | [symbol] | [symbol] | [symbol] | [symbol] |
| 38 | [symbol] | Cytarabine | [symbol] | [symbol] | [symbol] | [symbol] | [symbol] |
| 39 | [symbol] | Cytarabine | [symbol] | [symbol] | [symbol] | [symbol] | [symbol] |
| 40 | [symbol] | Cytarabine | [symbol] | [symbol] | [symbol] | [symbol] | [symbol] |
| 41 | [symbol] | Cytarabine | [symbol] | [symbol] | [symbol] | [symbol] | [symbol] |
| 42 | [symbol] | Cytarabine | [symbol] | [symbol] | [symbol] | [symbol] | [symbol] |
| 43 | [symbol] | Cytarabine | N/A | N/A | N/A | N/A | [symbol] |
| 44 | [symbol] | Cytarabine | [symbol] | [symbol] | [symbol] | [symbol] | [symbol] |
| 45 | [symbol] | Cytarabine | [symbol] | [symbol] | [symbol] | [symbol] | [symbol] |
| 46 | [symbol] | Cytarabine | [symbol] | [symbol] | [symbol] | [symbol] | [symbol] |
| 47 | [symbol] | Cytarabine | | [symbol] | [symbol] | [symbol] | [symbol] |
| 48 | [symbol] | Cytarabine | [symbol] | [symbol] | [symbol] | [symbol] | [symbol] |
| 49 | [symbol] | Cytarabine | | [symbol] | | [symbol] | [symbol] |
| 50 | [symbol] | Cytarabine | [symbol] | N/A | N/A | N/A | [symbol] |
| 51 | [symbol] | Cytarabine | | N/A | N/A | N/A | [symbol] |
| 52 | [symbol] | Cytarabine | | [symbol] | [symbol] | [symbol] | [symbol] |
| 53 | [symbol] | Cytarabine | [symbol] | [symbol] | [symbol] | [symbol] | [symbol] |
| 54 | [symbol] | Cytarabine | | [symbol] | [symbol] | [symbol] | [symbol] |
| 55 | [symbol] | Cytarabine | [symbol] | [symbol] | [symbol] | [symbol] | [symbol] |
| 56 | [symbol] | Cytarabine | [symbol] | [symbol] | [symbol] | [symbol] | [symbol] |
| 57 | [symbol] | Cytarabine | [symbol] | [symbol] | [symbol] | [symbol] | [symbol] |
| 58 | [symbol] | Cytarabine | [symbol] | [symbol] | [symbol] | [symbol] | [symbol] |
| 59 | [symbol] | Cytarabine | [symbol] | [symbol] | [symbol] | [symbol] | [symbol] |
| 60 | [symbol] | Cytarabine | [symbol] | [symbol] | [symbol] | [symbol] | [symbol] |
| 61 | [symbol] | Cytarabine | [symbol] | [symbol] | [symbol] | [symbol] | [symbol] |
| 62 | [symbol] | Cytarabine | [symbol] | [symbol] | [symbol] | [symbol] | [symbol] |
| 63 | [symbol] | Cytarabine | [symbol] | [symbol] | [symbol] | [symbol] | [symbol] |
| 64 | [symbol] | Cytarabine | [symbol] | [symbol] | [symbol] | [symbol] | [symbol] |
| 65 | [symbol] | Cytarabine | [symbol] | [symbol] | [symbol] | [symbol] | [symbol] |
| 66 | [symbol] | Cytarabine | [symbol] | [symbol] | [symbol] | [symbol] | [symbol] |
| 67 | [symbol] | Cytarabine | [symbol] | [symbol] | [symbol] | [symbol] | [symbol] |
| 68 | [symbol] | Cytarabine | [symbol] | [symbol] | [symbol] | [symbol] | [symbol] |
| 69 | [symbol] | Cytarabine | | N/A | N/A | N/A | [symbol] |

[symbol]

indicates data missing or illegible when filed

TABLE 3
Doxorubicin Single agent response profile. Table describing
IC50, EC50, GI50 and Max response for the panel of 69
cell lines treated with doxorubicin for 96 hours.

| | Cell | | | | | Max | Number |
|---|---|---|---|---|---|---|---|
| | Line | Compound | IC50 | EC50 | GI50 | Response | of Rep- |
| Count | Name | Name | [symbol] | [symbol] | [symbol] | [symbol] | licates |
| 70 | [symbol] | Doxorubicin | [symbol] | [symbol] | [symbol] | [symbol] | [symbol] |
| | | [symbol] | | | | | |
| 71 | [symbol] | Doxorubicin | [symbol] | [symbol] | [symbol] | [symbol] | [symbol] |
| | | [symbol] | | | | | |
| 72 | [symbol] | Doxorubicin | [symbol] | [symbol] | [symbol] | [symbol] | [symbol] |
| | | [symbol] | | | | | |
| 73 | [symbol] | Doxorubicin | [symbol] | [symbol] | [symbol] | [symbol] | [symbol] |
| | | [symbol] | | | | | |
| 74 | [symbol] | Doxorubicin | [symbol] | [symbol] | [symbol] | [symbol] | [symbol] |
| | | [symbol] | | | | | |
| 75 | [symbol] | Doxorubicin | [symbol] | [symbol] | [symbol] | [symbol] | [symbol] |
| | | [symbol] | | | | | |
| 76 | [symbol] | Doxorubicin | [symbol] | [symbol] | [symbol] | [symbol] | [symbol] |
| | | [symbol] | | | | | |
| 77 | [symbol] | Doxorubicin | [symbol] | [symbol] | [symbol] | [symbol] | [symbol] |
| | | [symbol] | | | | | |
| 78 | [symbol] | Doxorubicin | [symbol] | [symbol] | [symbol] | [symbol] | [symbol] |
| | | [symbol] | | | | | |
| 79 | [symbol] | Doxorubicin | [symbol] | [symbol] | [symbol] | [symbol] | [symbol] |
| | | [symbol] | | | | | |
| 80 | [symbol] | Doxorubicin | [symbol] | [symbol] | [symbol] | [symbol] | [symbol] |
| | | [symbol] | | | | | |
| 81 | [symbol] | Doxorubicin | [symbol] | [symbol] | [symbol] | [symbol] | [symbol] |
| | | [symbol] | | | | | |
| 82 | [symbol] | Doxorubicin | [symbol] | N/A | N/A | N/A | [symbol] |
| | | [symbol] | | | | | |
| 83 | [symbol] | Doxorubicin | [symbol] | [symbol] | [symbol] | [symbol] | [symbol] |
| | | [symbol] | | | | | |
| 84 | [symbol] | Doxorubicin | [symbol] | [symbol] | [symbol] | [symbol] | [symbol] |
| | | [symbol] | | | | | |
| 85 | [symbol] | Doxorubicin | [symbol] | [symbol] | [symbol] | [symbol] | [symbol] |
| | | [symbol] | | | | | |
| 86 | [symbol] | Doxorubicin | [symbol] | [symbol] | [symbol] | [symbol] | [symbol] |
| | | [symbol] | | | | | |
| 87 | [symbol] | Doxorubicin | [symbol] | [symbol] | [symbol] | [symbol] | [symbol] |
| | | [symbol] | | | | | |
| 88 | [symbol] | Doxorubicin | [symbol] | [symbol] | [symbol] | [symbol] | [symbol] |
| | | [symbol] | | | | | |
| 89 | [symbol] | Doxorubicin | [symbol] | [symbol] | [symbol] | [symbol] | [symbol] |
| | | [symbol] | | | | | |
| 90 | [symbol] | Doxorubicin | [symbol] | [symbol] | [symbol] | [symbol] | [symbol] |
| | | [symbol] | | | | | |
| 91 | [symbol] | Doxorubicin | [symbol] | [symbol] | [symbol] | [symbol] | [symbol] |
| | | [symbol] | | | | | |
| 92 | [symbol] | Doxorubicin | [symbol] | [symbol] | [symbol] | [symbol] | [symbol] |
| | | [symbol] | | | | | |
| 93 | [symbol] | Doxorubicin | [symbol] | N/A | N/A | N/A | [symbol] |
| | | [symbol] | | | | | |
| 94 | [symbol] | Doxorubicin | [symbol] | [symbol] | [symbol] | [symbol] | [symbol] |
| | | [symbol] | | | | | |
| 95 | [symbol] | Doxorubicin | [symbol] | [symbol] | [symbol] | [symbol] | [symbol] |
| | | [symbol] | | | | | |
| 96 | [symbol] | Doxorubicin | [symbol] | [symbol] | [symbol] | [symbol] | [symbol] |
| | | [symbol] | | | | | |
| 97 | [symbol] | Doxorubicin | [symbol] | [symbol] | [symbol] | [symbol] | [symbol] |
| | | [symbol] | | | | | |
| 98 | [symbol] | Doxorubicin | [symbol] | N/A | N/A | N/A | [symbol] |
| | | [symbol] | | | | | |
| 99 | [symbol] | Doxorubicin | [symbol] | [symbol] | [symbol] | [symbol] | [symbol] |
| | | [symbol] | | | | | |
| 100 | [symbol] | Doxorubicin | [symbol] | [symbol] | [symbol] | [symbol] | [symbol] |
| | | [symbol] | | | | | |
| 101 | [symbol] | Doxorubicin | [symbol] | [symbol] | [symbol] | [symbol] | [symbol] |
| | | [symbol] | | | | | |
| 102 | [symbol] | Doxorubicin | [symbol] | [symbol] | [symbol] | [symbol] | [symbol] |
| | | [symbol] | | | | | |
| 103 | [symbol] | Doxorubicin | [symbol] | [symbol] | [symbol] | [symbol] | [symbol] |
| | | [symbol] | | | | | |
| 104 | [symbol] | Doxorubicin | [symbol] | [symbol] | [symbol] | [symbol] | [symbol] |
| | | [symbol] | | | | | |
| 105 | [symbol] | Doxorubicin | [symbol] | [symbol] | [symbol] | [symbol] | [symbol] |
| | | [symbol] | | | | | |
| 106 | [symbol] | Doxorubicin | [symbol] | [symbol] | [symbol] | [symbol] | [symbol] |
| | | [symbol] | | | | | |
| 107 | [symbol] | Doxorubicin | [symbol] | [symbol] | [symbol] | [symbol] | [symbol] |
| | | [symbol] | | | | | |
| 108 | [symbol] | Doxorubicin | [symbol] | [symbol] | [symbol] | [symbol] | [symbol] |
| | | [symbol] | | | | | |
| 109 | [symbol] | Doxorubicin | [symbol] | [symbol] | [symbol] | [symbol] | [symbol] |
| | | [symbol] | | | | | |
| 110 | [symbol] | Doxorubicin | [symbol] | [symbol] | [symbol] | [symbol] | [symbol] |
| | | [symbol] | | | | | |
| 111 | [symbol] | Doxorubicin | [symbol] | [symbol] | [symbol] | [symbol] | [symbol] |
| | | [symbol] | | | | | |
| 112 | [symbol] | Doxorubicin | [symbol] | N/A | N/A | N/A | [symbol] |
| | | [symbol] | | | | | |
| 113 | [symbol] | Doxorubicin | [symbol] | [symbol] | [symbol] | [symbol] | [symbol] |
| | | [symbol] | | | | | |
| 114 | [symbol] | Doxorubicin | [symbol] | [symbol] | [symbol] | [symbol] | [symbol] |
| | | [symbol] | | | | | |
| 115 | [symbol] | Doxorubicin | [symbol] | [symbol] | [symbol] | [symbol] | [symbol] |
| | | [symbol] | | | | | |
| 116 | [symbol] | Doxorubicin | [symbol] | [symbol] | [symbol] | [symbol] | [symbol] |
| | | [symbol] | | | | | |
| 117 | [symbol] | Doxorubicin | [symbol] | [symbol] | [symbol] | [symbol] | [symbol] |
| | | [symbol] | | | | | |
| 118 | [symbol] | Doxorubicin | [symbol] | [symbol] | [symbol] | [symbol] | [symbol] |
| | | [symbol] | | | | | |
| 119 | [symbol] | Doxorubicin | [symbol] | N/A | N/A | N/A | [symbol] |
| | | [symbol] | | | | | |
| 120 | [symbol] | Doxorubicin | [symbol] | N/A | N/A | N/A | [symbol] |
| | | [symbol] | | | | | |
| 121 | [symbol] | Doxorubicin | [symbol] | [symbol] | [symbol] | [symbol] | [symbol] |
| | | [symbol] | | | | | |
| 122 | [symbol] | Doxorubicin | [symbol] | [symbol] | [symbol] | [symbol] | [symbol] |
| | | [symbol] | | | | | |
| 123 | [symbol] | Doxorubicin | [symbol] | [symbol] | [symbol] | [symbol] | [symbol] |
| | | [symbol] | | | | | |
| 124 | [symbol] | Doxorubicin | [symbol] | [symbol] | [symbol] | [symbol] | [symbol] |
| | | [symbol] | | | | | |
| 125 | [symbol] | Doxorubicin | [symbol] | [symbol] | [symbol] | [symbol] | [symbol] |
| | | [symbol] | | | | | |
| 126 | [symbol] | Doxorubicin | [symbol] | [symbol] | [symbol] | [symbol] | [symbol] |
| | | [symbol] | | | | | |
| 127 | [symbol] | Doxorubicin | [symbol] | [symbol] | [symbol] | [symbol] | [symbol] |
| | | [symbol] | | | | | |
| 128 | [symbol] | Doxorubicin | [symbol] | [symbol] | [symbol] | [symbol] | [symbol] |
| | | [symbol] | | | | | |
| 129 | [symbol] | Doxorubicin | [symbol] | [symbol] | [symbol] | [symbol] | [symbol] |
| | | [symbol] | | | | | |
| 130 | [symbol] | Doxorubicin | [symbol] | [symbol] | [symbol] | [symbol] | [symbol] |
| | | [symbol] | | | | | |
| 131 | [symbol] | Doxorubicin | [symbol] | [symbol] | [symbol] | [symbol] | [symbol] |
| | | [symbol] | | | | | |
| 132 | [symbol] | Doxorubicin | [symbol] | [symbol] | [symbol] | [symbol] | [symbol] |
| | | [symbol] | | | | | |
| 133 | [symbol] | Doxorubicin | [symbol] | [symbol] | [symbol] | [symbol] | [symbol] |
| | | [symbol] | | | | | |
| 134 | [symbol] | Doxorubicin | [symbol] | [symbol] | [symbol] | [symbol] | [symbol] |
| | | [symbol] | | | | | |
| 135 | [symbol] | Doxorubicin | [symbol] | [symbol] | [symbol] | [symbol] | [symbol] |
| | | [symbol] | | | | | |
| 136 | [symbol] | Doxorubicin | [symbol] | [symbol] | [symbol] | [symbol] | [symbol] |
| | | [symbol] | | | | | |
| 137 | [symbol] | Doxorubicin | [symbol] | [symbol] | [symbol] | [symbol] | [symbol] |
| | | [symbol] | | | | | |
| 138 | [symbol] | Doxorubicin | [symbol] | N/A | N/A | N/A | [symbol] |
| | | [symbol] | | | | | |

[symbol]

indicates data missing or illegible when filed

TABLE 4
Zelenistat Single agent response profile. Table describing
IC50, EC50, GI50 and Max response for the panel of 69
cell lines treated with zelenirstat for 96 hours.

| | Cell | | | | | Max | Number |
|---|---|---|---|---|---|---|---|
| | Line | Compound | IC50 | EC50 | GI50 | Response | of Rep- |
| Count | Name | Name | [symbol] | [symbol] | [symbol] | [symbol] | licates |
| 139 | [symbol] | [symbol] | [symbol] | [symbol] | [symbol] | [symbol] | [symbol] |
| 140 | [symbol] | [symbol] | [symbol] | [symbol] | [symbol] | [symbol] | [symbol] |
| 141 | [symbol] | [symbol] | [symbol] | [symbol] | [symbol] | [symbol] | [symbol] |
| 142 | [symbol] | [symbol] | [symbol] | [symbol] | [symbol] | [symbol] | [symbol] |
| 143 | [symbol] | [symbol] | [symbol] | [symbol] | [symbol] | [symbol] | [symbol] |
| 144 | [symbol] | [symbol] | [symbol] | [symbol] | [symbol] | [symbol] | [symbol] |
| 145 | [symbol] | [symbol] | [symbol] | [symbol] | [symbol] | [symbol] | [symbol] |
| 146 | [symbol] | [symbol] | [symbol] | [symbol] | [symbol] | [symbol] | [symbol] |
| 147 | [symbol] | [symbol] | [symbol] | [symbol] | [symbol] | [symbol] | [symbol] |
| 148 | [symbol] | [symbol] | [symbol] | [symbol] | [symbol] | [symbol] | [symbol] |
| 149 | [symbol] | [symbol] | [symbol] | [symbol] | [symbol] | [symbol] | [symbol] |
| 150 | [symbol] | [symbol] | [symbol] | [symbol] | [symbol] | [symbol] | [symbol] |
| 151 | [symbol] | [symbol] | [symbol] | N/A | N/A | N/A | [symbol] |
| 152 | [symbol] | [symbol] | [symbol] | [symbol] | [symbol] | [symbol] | [symbol] |
| 153 | [symbol] | [symbol] | [symbol] | [symbol] | [symbol] | [symbol] | [symbol] |
| 154 | [symbol] | [symbol] | | [symbol] | | [symbol] | [symbol] |
| 155 | [symbol] | [symbol] | [symbol] | [symbol] | [symbol] | [symbol] | [symbol] |
| 156 | [symbol] | [symbol] | [symbol] | [symbol] | [symbol] | [symbol] | [symbol] |
| 157 | [symbol] | [symbol] | [symbol] | [symbol] | [symbol] | [symbol] | [symbol] |
| 158 | [symbol] | [symbol] | [symbol] | [symbol] | [symbol] | [symbol] | [symbol] |
| 159 | [symbol] | [symbol] | [symbol] | [symbol] | [symbol] | [symbol] | [symbol] |
| 160 | [symbol] | [symbol] | [symbol] | [symbol] | [symbol] | [symbol] | [symbol] |
| 161 | [symbol] | [symbol] | [symbol] | [symbol] | [symbol] | [symbol] | [symbol] |
| 162 | [symbol] | [symbol] | | N/A | N/A | N/A | [symbol] |
| 163 | [symbol] | [symbol] | [symbol] | [symbol] | [symbol] | [symbol] | [symbol] |
| 164 | [symbol] | [symbol] | [symbol] | [symbol] | [symbol] | [symbol] | [symbol] |
| 165 | [symbol] | [symbol] | [symbol] | [symbol] | [symbol] | [symbol] | [symbol] |
| 166 | [symbol] | [symbol] | [symbol] | [symbol] | [symbol] | [symbol] | [symbol] |
| 167 | [symbol] | [symbol] | [symbol] | N/A | N/A | N/A | [symbol] |
| 168 | [symbol] | [symbol] | [symbol] | [symbol] | [symbol] | [symbol] | [symbol] |
| 169 | [symbol] | [symbol] | [symbol] | [symbol] | [symbol] | [symbol] | [symbol] |
| 170 | [symbol] | [symbol] | [symbol] | [symbol] | [symbol] | [symbol] | [symbol] |
| 171 | [symbol] | [symbol] | [symbol] | [symbol] | [symbol] | [symbol] | [symbol] |
| 172 | [symbol] | [symbol] | [symbol] | [symbol] | [symbol] | [symbol] | [symbol] |
| 173 | [symbol] | [symbol] | [symbol] | [symbol] | [symbol] | [symbol] | [symbol] |
| 174 | [symbol] | [symbol] | [symbol] | [symbol] | [symbol] | [symbol] | [symbol] |
| 175 | [symbol] | [symbol] | [symbol] | [symbol] | [symbol] | [symbol] | [symbol] |
| 176 | [symbol] | [symbol] | [symbol] | [symbol] | [symbol] | [symbol] | [symbol] |
| 177 | [symbol] | [symbol] | [symbol] | [symbol] | [symbol] | [symbol] | [symbol] |
| 178 | [symbol] | [symbol] | [symbol] | [symbol] | [symbol] | [symbol] | [symbol] |
| 179 | [symbol] | [symbol] | [symbol] | [symbol] | [symbol] | [symbol] | [symbol] |
| 180 | [symbol] | [symbol] | [symbol] | [symbol] | [symbol] | [symbol] | [symbol] |
| 181 | [symbol] | [symbol] | | N/A | N/A | N/A | [symbol] |
| 182 | [symbol] | [symbol] | [symbol] | [symbol] | [symbol] | [symbol] | [symbol] |
| 183 | [symbol] | [symbol] | | [symbol] | [symbol] | [symbol] | [symbol] |
| 184 | [symbol] | [symbol] | [symbol] | [symbol] | [symbol] | [symbol] | [symbol] |
| 185 | [symbol] | [symbol] | | [symbol] | | [symbol] | [symbol] |
| 186 | [symbol] | [symbol] | [symbol] | [symbol] | [symbol] | [symbol] | [symbol] |
| 187 | [symbol] | [symbol] | [symbol] | [symbol] | [symbol] | [symbol] | [symbol] |
| 188 | [symbol] | [symbol] | [symbol] | N/A | N/A | N/A | [symbol] |
| 189 | [symbol] | [symbol] | [symbol] | N/A | N/A | N/A | [symbol] |
| 190 | [symbol] | [symbol] | [symbol] | [symbol] | [symbol] | [symbol] | [symbol] |
| 191 | [symbol] | [symbol] | [symbol] | [symbol] | [symbol] | [symbol] | [symbol] |
| 192 | [symbol] | [symbol] | [symbol] | [symbol] | [symbol] | [symbol] | [symbol] |
| 193 | [symbol] | [symbol] | [symbol] | [symbol] | [symbol] | [symbol] | [symbol] |
| 194 | [symbol] | [symbol] | [symbol] | [symbol] | [symbol] | [symbol] | [symbol] |
| 195 | [symbol] | [symbol] | [symbol] | [symbol] | [symbol] | [symbol] | [symbol] |
| 196 | [symbol] | [symbol] | [symbol] | [symbol] | [symbol] | [symbol] | [symbol] |
| 197 | [symbol] | [symbol] | [symbol] | [symbol] | [symbol] | [symbol] | [symbol] |
| 198 | [symbol] | [symbol] | [symbol] | [symbol] | [symbol] | [symbol] | [symbol] |
| 199 | [symbol] | [symbol] | [symbol] | [symbol] | [symbol] | [symbol] | [symbol] |
| 200 | [symbol] | [symbol] | [symbol] | [symbol] | [symbol] | [symbol] | [symbol] |
| 201 | [symbol] | [symbol] | [symbol] | [symbol] | [symbol] | [symbol] | [symbol] |
| 202 | [symbol] | [symbol] | [symbol] | [symbol] | [symbol] | [symbol] | [symbol] |
| 203 | [symbol] | [symbol] | [symbol] | [symbol] | [symbol] | [symbol] | [symbol] |
| 204 | [symbol] | [symbol] | | [symbol] | | [symbol] | [symbol] |
| 205 | [symbol] | [symbol] | [symbol] | [symbol] | [symbol] | [symbol] | [symbol] |
| 206 | [symbol] | [symbol] | | [symbol] | [symbol] | [symbol] | [symbol] |
| 207 | [symbol] | [symbol] | | N/A | N/A | N/A | [symbol] |

[symbol]

indicates data missing or illegible when filed

FIG. 2 shows a comparison of the EC50 for the panel of 69 cell lines treated with either cytarabine, doxorubicin or zelenirstat.

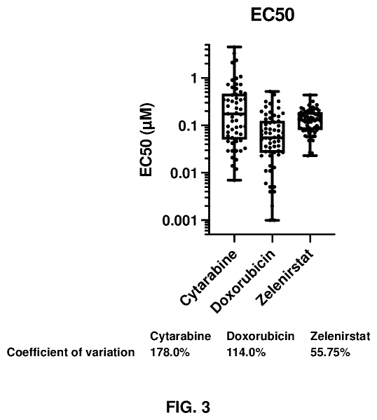

FIG. 3. shows a distribution of EC50 for the panel of 69 cell lines treated with either cytarabine, doxorubicin or zelenirstat. EC50 variation for the cells treated with cell lines is lower than the EC50 variation for the cells treated with either cytarabine or doxorubicin. Zelenirstat may target efficiently a wider range of cancer cell lines due to its impact on several myristoylated proteins. The coefficient of variation (CV), also known as “relative variability”, equals the standard deviation divided by the mean. It can be expressed either as a fraction or a percent.

### Combination Response Profile

TABLE 5
Zelenirstat × Cytarabine Combination response profile. Table describing
best CI, bliss volume, Lowe volume and synergy score for the panel of 69 cell
lines treated with combination of zelenirstat and cytarabine for 96 hours.

| | Cell | | | | | | | | |
|---|---|---|---|---|---|---|---|---|---|
| | [symbol] | Combination | Best | Best [symbol] | [symbol] | [symbol] | [symbol] | Synergy | Number of |
| Count | Name | Name | [symbol] | Level [symbol] | Volume | Volume | Volume | Score | Replicates |
| 1 | [symbol] | [symbol] | [symbol] | [symbol] | [symbol] | [symbol] | [symbol] | [symbol] | [symbol] |
| 2 | [symbol] | [symbol] | [symbol] | [symbol] | [symbol] | [symbol] | [symbol] | [symbol] | [symbol] |
| 3 | [symbol] | [symbol] | [symbol] | [symbol] | [symbol] | [symbol] | [symbol] | [symbol] | [symbol] |
| 4 | [symbol] | [symbol] | [symbol] | [symbol] | [symbol] | [symbol] | [symbol] | [symbol] | [symbol] |
| 5 | [symbol] | [symbol] | [symbol] | [symbol] | [symbol] | [symbol] | [symbol] | [symbol] | [symbol] |
| 6 | [symbol] | [symbol] | [symbol] | [symbol] | [symbol] | [symbol] | [symbol] | [symbol] | [symbol] |
| 7 | [symbol] | [symbol] | [symbol] | [symbol] | [symbol] | [symbol] | [symbol] | [symbol] | [symbol] |
| 8 | [symbol] | [symbol] | [symbol] | [symbol] | [symbol] | [symbol] | [symbol] | [symbol] | [symbol] |
| 9 | [symbol] | [symbol] | [symbol] | [symbol] | [symbol] | [symbol] | [symbol] | [symbol] | [symbol] |
| 10 | [symbol] | [symbol] | [symbol] | [symbol] | [symbol] | [symbol] | [symbol] | [symbol] | [symbol] |
| 11 | [symbol] | [symbol] | [symbol] | [symbol] | [symbol] | [symbol] | [symbol] | [symbol] | [symbol] |
| 12 | [symbol] | [symbol] | [symbol] | [symbol] | [symbol] | [symbol] | [symbol] | [symbol] | [symbol] |
| 13 | [symbol] | [symbol] | N/A | N/A | N/A | N/A | N/A | N/A | [symbol] |
| 14 | [symbol] | [symbol] | [symbol] | [symbol] | [symbol] | [symbol] | [symbol] | [symbol] | [symbol] |
| 15 | [symbol] | [symbol] | [symbol] | [symbol] | [symbol] | [symbol] | [symbol] | [symbol] | [symbol] |
| 16 | [symbol] | [symbol] | [symbol] | [symbol] | [symbol] | [symbol] | [symbol] | [symbol] | [symbol] |
| 17 | [symbol] | [symbol] | [symbol] | [symbol] | [symbol] | [symbol] | [symbol] | [symbol] | [symbol] |
| 18 | [symbol] | [symbol] | [symbol] | [symbol] | [symbol] | [symbol] | [symbol] | [symbol] | [symbol] |
| 19 | [symbol] | [symbol] | [symbol] | [symbol] | [symbol] | [symbol] | [symbol] | [symbol] | [symbol] |
| 20 | [symbol] | [symbol] | [symbol] | [symbol] | [symbol] | [symbol] | [symbol] | [symbol] | [symbol] |
| 21 | [symbol] | [symbol] | [symbol] | [symbol] | [symbol] | [symbol] | [symbol] | [symbol] | [symbol] |
| 22 | [symbol] | [symbol] | [symbol] | [symbol] | [symbol] | [symbol] | [symbol] | [symbol] | [symbol] |
| 23 | [symbol] | [symbol] | [symbol] | [symbol] | [symbol] | [symbol] | [symbol] | [symbol] | [symbol] |
| 24 | [symbol] | [symbol] | N/A | N/A | N/A | N/A | N/A | N/A | [symbol] |
| 25 | [symbol] | [symbol] | [symbol] | [symbol] | [symbol] | [symbol] | [symbol] | [symbol] | [symbol] |
| 26 | [symbol] | [symbol] | [symbol] | [symbol] | [symbol] | [symbol] | [symbol] | [symbol] | [symbol] |
| 27 | [symbol] | [symbol] | [symbol] | [symbol] | [symbol] | [symbol] | [symbol] | [symbol] | [symbol] |
| 28 | [symbol] | [symbol] | [symbol] | [symbol] | [symbol] | [symbol] | [symbol] | [symbol] | [symbol] |
| 29 | [symbol] | [symbol] | N/A | N/A | N/A | N/A | N/A | N/A | [symbol] |
| 30 | [symbol] | [symbol] | [symbol] | [symbol] | [symbol] | [symbol] | [symbol] | [symbol] | [symbol] |
| 31 | [symbol] | [symbol] | [symbol] | [symbol] | [symbol] | [symbol] | [symbol] | [symbol] | [symbol] |
| 32 | [symbol] | [symbol] | [symbol] | [symbol] | [symbol] | [symbol] | [symbol] | [symbol] | [symbol] |
| 33 | [symbol] | [symbol] | [symbol] | [symbol] | [symbol] | [symbol] | [symbol] | [symbol] | [symbol] |
| 34 | [symbol] | [symbol] | [symbol] | [symbol] | [symbol] | [symbol] | [symbol] | [symbol] | [symbol] |
| 35 | [symbol] | [symbol] | [symbol] | [symbol] | [symbol] | [symbol] | [symbol] | [symbol] | [symbol] |
| 36 | [symbol] | [symbol] | [symbol] | [symbol] | [symbol] | [symbol] | [symbol] | [symbol] | [symbol] |
| 37 | [symbol] | [symbol] | [symbol] | [symbol] | [symbol] | [symbol] | [symbol] | [symbol] | [symbol] |
| 38 | [symbol] | [symbol] | [symbol] | [symbol] | [symbol] | [symbol] | [symbol] | [symbol] | [symbol] |
| 39 | [symbol] | [symbol] | [symbol] | [symbol] | [symbol] | [symbol] | [symbol] | [symbol] | [symbol] |
| 40 | [symbol] | [symbol] | [symbol] | [symbol] | [symbol] | [symbol] | [symbol] | [symbol] | [symbol] |
| 41 | [symbol] | [symbol] | [symbol] | [symbol] | [symbol] | [symbol] | [symbol] | [symbol] | [symbol] |
| 42 | [symbol] | [symbol] | [symbol] | [symbol] | [symbol] | [symbol] | [symbol] | [symbol] | [symbol] |
| 43 | [symbol] | [symbol] | N/A | N/A | N/A | N/A | N/A | N/A | [symbol] |
| 44 | [symbol] | [symbol] | [symbol] | [symbol] | [symbol] | [symbol] | [symbol] | [symbol] | [symbol] |
| 45 | [symbol] | [symbol] | [symbol] | [symbol] | [symbol] | [symbol] | [symbol] | [symbol] | [symbol] |
| 46 | [symbol] | [symbol] | [symbol] | [symbol] | [symbol] | [symbol] | [symbol] | [symbol] | [symbol] |
| 47 | [symbol] | [symbol] | [symbol] | [symbol] | [symbol] | [symbol] | [symbol] | [symbol] | [symbol] |
| 48 | [symbol] | [symbol] | [symbol] | [symbol] | [symbol] | [symbol] | [symbol] | [symbol] | [symbol] |
| 49 | [symbol] | [symbol] | [symbol] | [symbol] | [symbol] | [symbol] | [symbol] | [symbol] | [symbol] |
| 50 | [symbol] | [symbol] | N/A | N/A | N/A | N/A | N/A | N/A | [symbol] |
| 51 | [symbol] | [symbol] | N/A | N/A | N/A | N/A | N/A | N/A | [symbol] |
| 52 | [symbol] | [symbol] | [symbol] | [symbol] | [symbol] | [symbol] | [symbol] | [symbol] | [symbol] |
| 53 | [symbol] | [symbol] | [symbol] | [symbol] | [symbol] | [symbol] | [symbol] | [symbol] | [symbol] |
| 54 | [symbol] | [symbol] | [symbol] | [symbol] | [symbol] | [symbol] | [symbol] | [symbol] | [symbol] |
| 55 | [symbol] | [symbol] | [symbol] | [symbol] | [symbol] | [symbol] | [symbol] | [symbol] | [symbol] |
| 56 | [symbol] | [symbol] | [symbol] | [symbol] | [symbol] | [symbol] | [symbol] | [symbol] | [symbol] |
| 57 | [symbol] | [symbol] | [symbol] | [symbol] | [symbol] | [symbol] | [symbol] | [symbol] | [symbol] |
| 58 | [symbol] | [symbol] | [symbol] | [symbol] | [symbol] | [symbol] | [symbol] | [symbol] | [symbol] |
| 59 | [symbol] | [symbol] | [symbol] | [symbol] | [symbol] | [symbol] | [symbol] | [symbol] | [symbol] |
| 60 | [symbol] | [symbol] | [symbol] | [symbol] | [symbol] | [symbol] | [symbol] | [symbol] | [symbol] |
| 61 | [symbol] | [symbol] | [symbol] | [symbol] | [symbol] | [symbol] | [symbol] | [symbol] | [symbol] |
| 62 | [symbol] | [symbol] | [symbol] | [symbol] | [symbol] | [symbol] | [symbol] | [symbol] | [symbol] |
| 63 | [symbol] | [symbol] | [symbol] | [symbol] | [symbol] | [symbol] | [symbol] | [symbol] | [symbol] |
| 64 | [symbol] | [symbol] | [symbol] | [symbol] | [symbol] | [symbol] | [symbol] | [symbol] | [symbol] |
| 65 | [symbol] | [symbol] | [symbol] | [symbol] | [symbol] | [symbol] | [symbol] | [symbol] | [symbol] |
| 66 | [symbol] | [symbol] | [symbol] | [symbol] | [symbol] | [symbol] | [symbol] | [symbol] | [symbol] |
| 67 | [symbol] | [symbol] | [symbol] | [symbol] | [symbol] | [symbol] | [symbol] | [symbol] | [symbol] |
| 68 | [symbol] | [symbol] | [symbol] | [symbol] | [symbol] | [symbol] | [symbol] | [symbol] | [symbol] |
| 69 | [symbol] | [symbol] | N/A | N/A | N/A | N/A | N/A | N/A | [symbol] |

[symbol]

indicates data missing or illegible when filed

TABLE 6
Zelenirstat × Doxorubicin Combination response profile. Table describing
best CI, bliss volume, Lowe volume and synergy score for the panel of 69 cell
lines treated with combination of zelenirstat and doxorubicin for 96 hours.

| | | | | Best | | | | | |
|---|---|---|---|---|---|---|---|---|---|
| | Cell | | | [symbol] | | | | | |
| | [symbol] | Combination | Best | Level | [symbol] | [symbol] | Lowe | Synergy | Number of |
| Count | Name | Name | [symbol] | [symbol] | volume | Volume | volume | Score | Replicates |
| 70 | [symbol] | [symbol] | [symbol] | [symbol] | [symbol] | [symbol] | [symbol] | [symbol] | [symbol] |
| 71 | [symbol] | [symbol] | [symbol] | [symbol] | [symbol] | [symbol] | [symbol] | [symbol] | [symbol] |
| 72 | [symbol] | [symbol] | [symbol] | [symbol] | [symbol] | [symbol] | [symbol] | [symbol] | [symbol] |
| 73 | [symbol] | [symbol] | [symbol] | [symbol] | [symbol] | [symbol] | [symbol] | [symbol] | [symbol] |
| 74 | [symbol] | [symbol] | [symbol] | [symbol] | [symbol] | [symbol] | [symbol] | [symbol] | [symbol] |
| 75 | [symbol] | [symbol] | [symbol] | [symbol] | [symbol] | [symbol] | [symbol] | [symbol] | [symbol] |
| 76 | [symbol] | [symbol] | [symbol] | [symbol] | [symbol] | [symbol] | [symbol] | [symbol] | [symbol] |
| 77 | [symbol] | [symbol] | [symbol] | [symbol] | [symbol] | [symbol] | [symbol] | [symbol] | [symbol] |
| 78 | [symbol] | [symbol] | [symbol] | [symbol] | [symbol] | [symbol] | [symbol] | [symbol] | [symbol] |
| 79 | [symbol] | [symbol] | [symbol] | [symbol] | [symbol] | [symbol] | [symbol] | [symbol] | [symbol] |
| 80 | [symbol] | [symbol] | [symbol] | [symbol] | [symbol] | [symbol] | [symbol] | [symbol] | [symbol] |
| 81 | [symbol] | [symbol] | [symbol] | [symbol] | [symbol] | [symbol] | [symbol] | [symbol] | [symbol] |
| 82 | [symbol] | [symbol] | [symbol] | [symbol] | [symbol] | [symbol] | [symbol] | [symbol] | [symbol] |
| 83 | [symbol] | [symbol] | [symbol] | [symbol] | [symbol] | [symbol] | [symbol] | [symbol] | [symbol] |
| 84 | [symbol] | [symbol] | [symbol] | [symbol] | [symbol] | [symbol] | [symbol] | [symbol] | [symbol] |
| 85 | [symbol] | [symbol] | [symbol] | [symbol] | [symbol] | [symbol] | [symbol] | [symbol] | [symbol] |
| 86 | [symbol] | [symbol] | [symbol] | [symbol] | [symbol] | [symbol] | [symbol] | [symbol] | [symbol] |
| 87 | [symbol] | [symbol] | [symbol] | [symbol] | [symbol] | [symbol] | [symbol] | [symbol] | [symbol] |
| 88 | [symbol] | [symbol] | [symbol] | [symbol] | [symbol] | [symbol] | [symbol] | [symbol] | [symbol] |
| 89 | [symbol] | [symbol] | [symbol] | [symbol] | [symbol] | [symbol] | [symbol] | [symbol] | [symbol] |
| 90 | [symbol] | [symbol] | | [symbol] | [symbol] | [symbol] | [symbol] | [symbol] | [symbol] |
| 91 | [symbol] | [symbol] | [symbol] | [symbol] | [symbol] | [symbol] | [symbol] | [symbol] | [symbol] |
| 92 | [symbol] | [symbol] | [symbol] | [symbol] | [symbol] | [symbol] | [symbol] | [symbol] | [symbol] |
| 93 | [symbol] | [symbol] | [symbol] | [symbol] | [symbol] | [symbol] | [symbol] | [symbol] | [symbol] |
| 94 | [symbol] | [symbol] | [symbol] | [symbol] | [symbol] | [symbol] | [symbol] | [symbol] | [symbol] |
| 95 | [symbol] | [symbol] | [symbol] | [symbol] | [symbol] | [symbol] | [symbol] | [symbol] | [symbol] |
| 96 | [symbol] | [symbol] | [symbol] | [symbol] | [symbol] | [symbol] | [symbol] | [symbol] | [symbol] |
| 97 | [symbol] | [symbol] | [symbol] | [symbol] | [symbol] | [symbol] | [symbol] | [symbol] | [symbol] |
| 98 | [symbol] | [symbol] | [symbol] | [symbol] | [symbol] | [symbol] | [symbol] | [symbol] | [symbol] |
| 99 | [symbol] | [symbol] | [symbol] | [symbol] | [symbol] | [symbol] | [symbol] | [symbol] | [symbol] |
| 100 | [symbol] | [symbol] | [symbol] | [symbol] | [symbol] | [symbol] | [symbol] | [symbol] | [symbol] |
| 101 | [symbol] | [symbol] | [symbol] | [symbol] | [symbol] | [symbol] | [symbol] | [symbol] | [symbol] |
| 102 | [symbol] | [symbol] | [symbol] | [symbol] | [symbol] | [symbol] | [symbol] | [symbol] | [symbol] |
| 103 | [symbol] | [symbol] | [symbol] | [symbol] | [symbol] | [symbol] | [symbol] | [symbol] | [symbol] |
| 104 | [symbol] | [symbol] | [symbol] | [symbol] | [symbol] | [symbol] | [symbol] | [symbol] | [symbol] |
| 105 | [symbol] | [symbol] | [symbol] | [symbol] | [symbol] | [symbol] | [symbol] | [symbol] | [symbol] |
| 106 | [symbol] | [symbol] | [symbol] | [symbol] | [symbol] | [symbol] | [symbol] | [symbol] | [symbol] |
| 107 | [symbol] | [symbol] | [symbol] | [symbol] | [symbol] | [symbol] | [symbol] | [symbol] | [symbol] |
| 108 | [symbol] | [symbol] | [symbol] | [symbol] | [symbol] | [symbol] | [symbol] | [symbol] | [symbol] |
| 109 | [symbol] | [symbol] | [symbol] | [symbol] | [symbol] | [symbol] | [symbol] | [symbol] | [symbol] |
| 110 | [symbol] | [symbol] | [symbol] | [symbol] | [symbol] | [symbol] | [symbol] | [symbol] | [symbol] |
| 111 | [symbol] | [symbol] | [symbol] | [symbol] | [symbol] | [symbol] | [symbol] | [symbol] | [symbol] |
| 112 | [symbol] | [symbol] | [symbol] | [symbol] | [symbol] | [symbol] | [symbol] | [symbol] | [symbol] |
| 113 | [symbol] | [symbol] | [symbol] | [symbol] | [symbol] | [symbol] | [symbol] | [symbol] | [symbol] |
| 114 | [symbol] | [symbol] | [symbol] | [symbol] | [symbol] | [symbol] | [symbol] | [symbol] | [symbol] |
| 115 | [symbol] | [symbol] | [symbol] | [symbol] | [symbol] | [symbol] | [symbol] | [symbol] | [symbol] |
| 116 | [symbol] | [symbol] | [symbol] | [symbol] | [symbol] | [symbol] | [symbol] | [symbol] | [symbol] |
| 117 | [symbol] | [symbol] | [symbol] | [symbol] | [symbol] | [symbol] | [symbol] | [symbol] | [symbol] |
| 118 | [symbol] | [symbol] | [symbol] | [symbol] | [symbol] | [symbol] | [symbol] | [symbol] | [symbol] |
| 119 | [symbol] | [symbol] | [symbol] | [symbol] | [symbol] | [symbol] | [symbol] | [symbol] | [symbol] |
| 120 | [symbol] | [symbol] | [symbol] | [symbol] | [symbol] | [symbol] | [symbol] | [symbol] | [symbol] |
| 121 | [symbol] | [symbol] | [symbol] | [symbol] | [symbol] | [symbol] | [symbol] | [symbol] | [symbol] |
| 122 | [symbol] | [symbol] | [symbol] | [symbol] | [symbol] | [symbol] | [symbol] | [symbol] | [symbol] |
| 123 | [symbol] | [symbol] | [symbol] | [symbol] | [symbol] | [symbol] | [symbol] | [symbol] | [symbol] |
| 124 | [symbol] | [symbol] | [symbol] | [symbol] | [symbol] | [symbol] | [symbol] | [symbol] | [symbol] |
| 125 | [symbol] | [symbol] | [symbol] | [symbol] | [symbol] | [symbol] | [symbol] | [symbol] | [symbol] |
| 126 | [symbol] | [symbol] | [symbol] | [symbol] | [symbol] | [symbol] | [symbol] | [symbol] | [symbol] |
| 127 | [symbol] | [symbol] | [symbol] | [symbol] | [symbol] | [symbol] | [symbol] | [symbol] | [symbol] |
| 128 | [symbol] | [symbol] | [symbol] | [symbol] | [symbol] | [symbol] | [symbol] | [symbol] | [symbol] |
| 129 | [symbol] | [symbol] | [symbol] | [symbol] | [symbol] | [symbol] | [symbol] | [symbol] | [symbol] |
| 130 | [symbol] | [symbol] | [symbol] | [symbol] | [symbol] | [symbol] | [symbol] | [symbol] | [symbol] |
| 131 | [symbol] | [symbol] | [symbol] | [symbol] | [symbol] | [symbol] | [symbol] | [symbol] | [symbol] |
| 132 | [symbol] | [symbol] | [symbol] | [symbol] | [symbol] | [symbol] | [symbol] | [symbol] | [symbol] |
| 133 | [symbol] | [symbol] | [symbol] | [symbol] | [symbol] | [symbol] | [symbol] | [symbol] | [symbol] |
| 134 | [symbol] | [symbol] | [symbol] | [symbol] | [symbol] | [symbol] | [symbol] | [symbol] | [symbol] |
| 135 | [symbol] | [symbol] | [symbol] | [symbol] | [symbol] | [symbol] | [symbol] | [symbol] | [symbol] |
| 136 | [symbol] | [symbol] | [symbol] | [symbol] | [symbol] | [symbol] | [symbol] | [symbol] | [symbol] |
| 137 | [symbol] | [symbol] | [symbol] | [symbol] | [symbol] | [symbol] | [symbol] | [symbol] | [symbol] |
| 138 | [symbol] | [symbol] | [symbol] | [symbol] | [symbol] | [symbol] | [symbol] | [symbol] | [symbol] |

[symbol]

indicates data missing or illegible when filed

TABLE 7
Global Heat Map of Combination Loewe Volume Scores with
Cell Lines Sorted by Tissue Type

| [symbol] | [symbol] | [symbol] | [symbol] |
|---|---|---|---|
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |

[symbol]

indicates data missing or illegible when filed

Using the matrix view in Chalice Analyzer, cell lines are sorted by tissue type and combinations are sorted by combination name. The Loewe Volume score is the displayed measure and the default Analyzer coloring indicates the strength of the combination interaction As described in the Methods, the Loewe Volume score is used to identify potential synergies (red) and potential antagonisms (blue). Potential moderate-to-strong synergies are highlighted in shades of pink to dark red. Potential antagonisms are highlighted in shades of blue to dark blue for potentially strong antagonistic interactions.

TABLE 8
Global Heat Map of Combination Synergy Scores with
Cell Lines Sorted by Tissue Type

| [symbol] | [symbol] | [symbol] | [symbol] |
|---|---|---|---|
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |

[symbol]

indicates data missing or illegible when filed

Using the matrix view in Chalice Analyzer, cell lines are sorted by tissue type, combinations are sorted by combination name and the Synergy Score measure is displayed. Combinations with high-to-moderate scores are individually reviewed to select a Synergy Score cutoff, using the histogram analysis function. This may facilitate highlighting of combination activities not particularly robust but potentially interesting. These moderate synergies can be the result of compound behavior in specific cell lines (for example, steep dose response(s) for enhancer or enhance or reduced compound activities linked to growth rate).

A Synergy Score cutoff of about 3.25 is made for this analysis. Combinations with a Synergy Score of less than 3.25 are shown in the table below in white. Potential moderate synergistic interactions (scores from about 3.25 to about 7.19) are shown in shades of pink and potential strong synergistic interactions (scores greater than 7.19) are shown in dark red.

The matrix view is invaluable for studying data trends, especially when working with large numbers of compounds or cell lines. However, definitive conclusions should be made only after careful examination of individual matrices. With consideration of these caveats, the matrix view can be used to quickly identify and assess combination effects for the cell line panel.

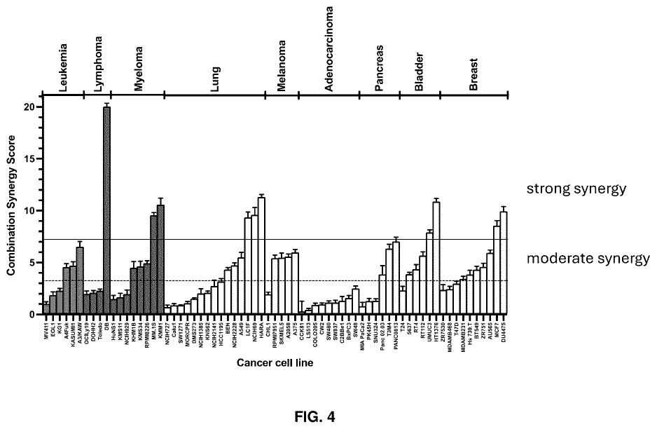

FIG. 4 shows cytarabine and Zelenirstat Combination Synergy Scores with Cell Lines Sorted by Tissue Type. Potential moderate synergistic interactions (scores from about 3.25 to about 7.19) and strong synergistic interactions (scores greater than about 7.19) are displayed on the histogram.

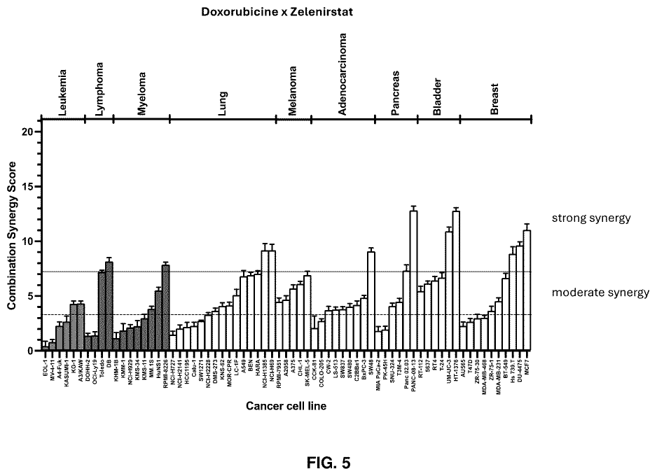

FIG. 5 shows doxorubicin and Zelenirstat Combination Synergy Scores with Cell Lines Sorted by Tissue Type. Potential moderate synergistic interactions (scores from about 3.25 to about 7.19) and strong synergistic interactions (scores greater than about 7.19) are displayed on the histogram.

### Results

A panel of 69 cancer cell lines (Table 1) demonstrates that all cells exhibit sensitivity to cytotoxic agents cytarabine, doxorubicin, and zelenirstat. Notably, hematologic cancer cell lines (Table 2-4; FIG. 2) exhibit heightened sensitivity to these agents. Furthermore, the variation in effective concentration (EC50) for zelenirstat is significantly lower compared to the observed variation for doxorubicin or cytarabine (FIG. 3).

Myristoylation remains an understudied protein modification, and the application of NMT inhibitors for cancer treatment, despite their promising potential, necessitates further investigation. Synergy combination screens were conducted to identify potential enhancers and cancer indications suitable for zelenirstat combination therapy.

Remarkably, about 65% (45/69) of tested cell lines exhibited moderate to strong synergy (13/69) between doxorubicin and zelenirstat, irrespective of the origin of cancer (hematological, lung, pancreas, bladder, and breast). Furthermore, cytarabine exhibited synergy in about 50% of the cell lines (34/69) when combined with zelenirstat, irrespective of the cancer origin (FIG. 5).

### DISCUSSION

Owing to its novel, unexplored, and multifaceted mechanism of action, zelenirstat, along with other myristoylation inhibitors, presents a promising avenue for exploring alternative drugs as potential synergistic enhancers of their efficacy when administered in combination.

Myristoylation is a lipid modification that facilitates the targeting of proteins, particularly members of the Src family kinases and other signaling molecules, to cellular membranes. By inhibiting myristoylation, these typically myristoylated proteins may fail to localize appropriately within the cell and may even be degraded by a dedicated glycine N-degron. This degradation may potentially diminish survival and proliferative signals in cancer cells. Doxorubicin induces DNA damage. If the cell's pro-survival signaling is compromised due to the mislocalization of non-functional or absent proteins, the cell may be less capable of mounting effective repair or anti-apoptotic responses. Consequently, doxorubicin's cytotoxicity may be amplified. Furthermore, both doxorubicin and NMTi are known to be apoptosis regulators^{38,39}, which may result in enhanced apoptotic responses when used in combination. In conclusion, the combined treatment (doxorubicin and zelenirstat) may overwhelm the cells' ability to cope with stress and damage, leading to enhanced apoptosis and tumor cell death.

Similarly, cytarabine may disrupt DNA synthesis during the S-phase. When the cell's survival signals are already compromised, the inability to effectively respond to the replication stress or DNA damage induced by cytarabine can tip the balance toward apoptosis. NMT inhibitors, such as zelenirstat, are also known to induce endoplasmic reticulum (ER) stress, cell cycle arrest, and apoptosis³⁸. This multifaceted stress may push cells beyond a critical threshold, leading to heightened apoptotic signaling and cell death.

### Example 2: Zelenirstat Combination with Dasatinib and Ibrutinib

In this example, cytotoxic activity of 2 combinations (Dasatinib & Zelenirstat and Ibrutinib & Zelenirstat) in lymphoma cell lines was tested. Zelenirstat (PCLX-001) may disrupt B-cell receptor signalling by targeting HGAL and Lyn myristoylation (their inhibition leading to the degradation of the proteins). In summary, upon BCR activation, myristoylated Lyn is recruited to lipid rafts, becomes activated, and phosphorylates BTK, while myristoylated HGAL is recruited and enhances signaling via SYK, and BTK. These events activate transcription factors such as c-Myc, P-ERK, NFκB, and CREB. PCLX-001 (zelenirstat) blocks the myristoylation of Lyn, HGAL, and Arf1, disrupting their membrane localization and calcium homeostasis, leading to their degradation and some of other key signaling proteins, increased ER stress, and ultimately apoptosis and cell death.

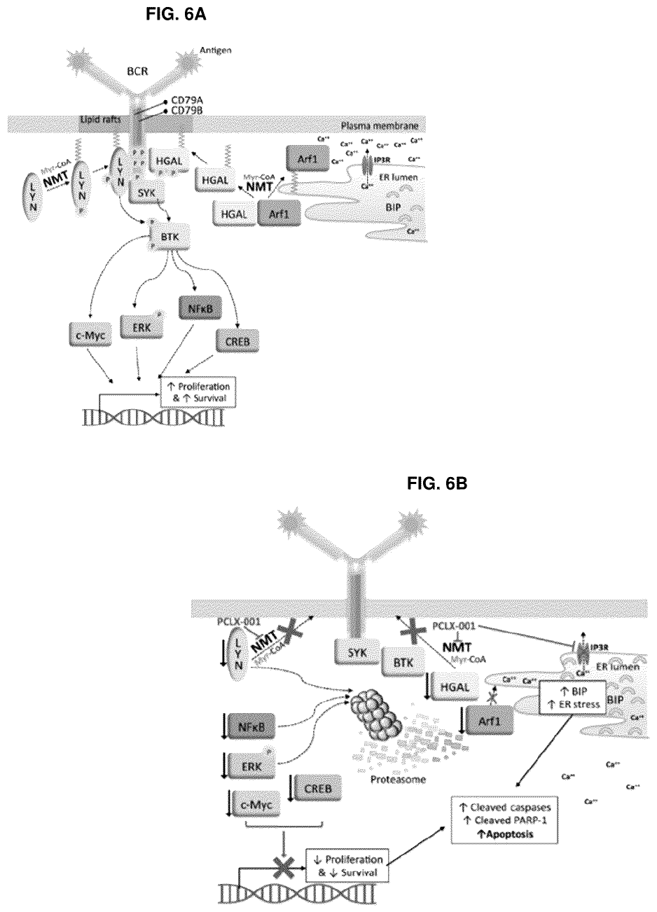

FIG. 6 illustrates a model depicting proposed PCLX-001 mechanism of action in B cell lymphoma. In this model, upon BCR activation, first the myristoylated SFK Lyn is recruited to the lipid raft domains of the plasma membrane containing the BCR, dephosphorylated Lyn at Y507 leads to its activation and autophosphorylation at Y396. This leads to the phosphorylation and activation of BTK at Y551 and Y223. Second, myristoylated HGAL is also recruited to the plasma membrane and phosphorylated thereby enhancing BCR signaling by stimulating SYK, BTK and the release of Ca⁺⁺ ions from the endoplasmic reticulum via the inositol-3-phosphate ion channel receptor (IP3R). Altogether these early signaling events lead to transcription activation by c-Myc, P-ERK, NFκB, and CREB. b The NMT inhibitor PCLX-001 prevents the myristoylation of Lyn-SFK (as well as other SFKs not shown in this model), HGAL and Arf1 thereby impeding the proper membrane targeting and function of these proteins. PCLX-001 treatment impedes calcium homeostasis by reducing the BCR mediated Ca-release from the ER and increasing basal Ca⁺⁺ levels in cells in addition to promote the degradation of both myristoylated (Lyn, HGAL, Arf1) and, surprisingly, non-myristoylated proteins (NFκB, P-ERK, c-Myc and CREB), some via the ubiquitin-proteasome pathway thereby further abrogating downstream BCR signaling and increasing ER stress leading to apoptosis and cell death.

Ibrutinib is an irreversible inhibitor of Bruton's tyrosine kinase (BTK). It covalently binds to the cysteine residue (Cys481) in the active site of BTK, thereby disrupting the B-cell receptor (BCR) signaling pathway. This disruption is pivotal for the survival, proliferation, and migration of malignant B-cells, leading to diminished survival signals and the induction of apoptosis in lymphoma cells, particularly in B-cell malignancies such as mantle cell lymphoma and chronic lymphocytic leukemia. In contrast, dasatinib is a multi-kinase inhibitor that targets multiple tyrosine kinases, including BCR-ABL and the Src family kinases (SFKs). In the context of lymphoma, dasatinib's inhibition of SFKs, such as Lyn, interferes with pivotal signaling pathways that promote cell growth and survival. By simultaneously targeting multiple kinases involved in malignant cell proliferation, dasatinib effectively suppresses tumor growth and induces apoptosis, contributing to its efficacy in lymphoma treatment.

Multiple analogous key proteins involved in the pro-survival B-cell receptor (BCR) signaling pathway are targeted by zelenirstat, ibrutinib, and dasatinib.

### Method

Viability of Cells Treated with PCLX-001, Dasatinib, and Ibrutinib

IM9 and BL2 (1×10⁵ cells) were grown in six-well plates in 4 ml media/well and incubated with increasing concentrations of PCLX-001, dasatinib, and ibrutinib for up to 96 h. Viability of cells treated with PCLX-001 was measured by calcein AM staining (Life Technologies) according to the manufacturer's instructions on a Cytation 5 plate reader (Biotek, Winooski, VT). Calcein assay consists of measuring the cell viability ratio (live cells/total cells and expressed as % viability). Cells were stained with the Nuclear-ID Blue/Red cell viability reagent (GFP-certified, Enzo Life Sciences) to identify total cells, and dead cells while live cells were stained with Calcein AM (Life Technologies) according to manufacturer's instructions. Cell count was performed using a Cytation 5 Cell Imaging Multi-Mode Reader (Biotek Instruments, Inc.) and analyzed by Biotek Gen5 Data Analysis software (version 2.09).

FIG. 6. Dasatinib and ibrutinib do not synergize the cytotoxic effects of PCLX-001 in IM9 and BL2 cells. IM9 (A) and BL2 (B) cells were incubated with 0.01, 0.1 and 1 μM PCLX-001 in combination with 0.1 and 1 μM dasatinib or ibrutinib for 96 hours. No additive or synergistic effects were observed upon the addition of dasatinib or ibrutinib to PCLX-001. As seen throughout our experiments, malignant BL2 cells are more sensitive to PCLX-001 than normal IM9 B cells. Cell viability was measured using calcein assay and represents an average of three independent experiments. Errors bars depict s.e.m.

### Results

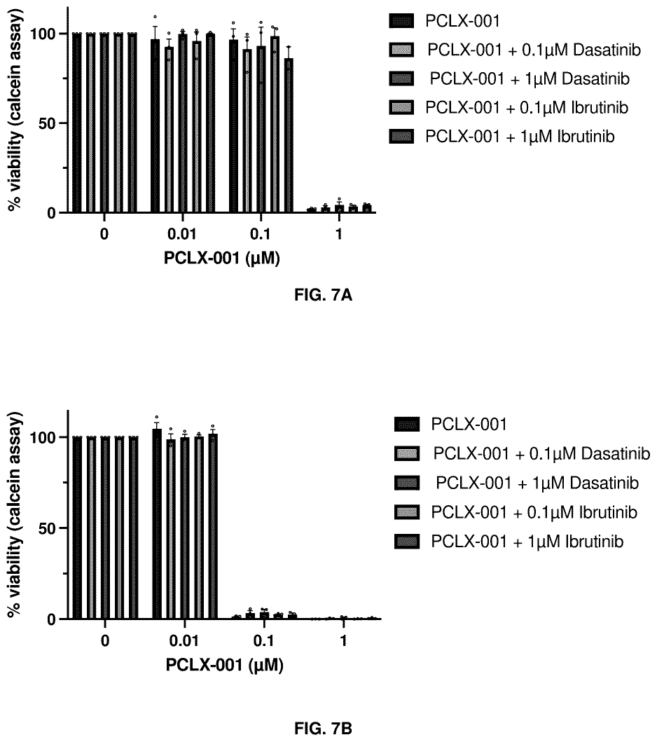

According to this example, dasatinib and ibrutinib do not synergize the cytotoxic effects of PCLX-001 in lymphoma cells (FIG. 7). The viability of BL2 (Burkitt Lymphoma) and IM9 (normal lymphocytes) cells is not affected by the addition of dasatinib or ibrutinib (up to 1 uM) when they are already treated with PCLX-001. Furthermore, it is noteworthy that cancerous BL2 cells exhibit greater sensitivity to zelenristat treatment compared to normal lymphocyte cell line IM9.

### DISCUSSION

The combination treatment of either dasatinib or ibrutinib at concentrations of 0.1 and 1.0 μM to PCLX-001 at 0.01, 0.1, and 1.0 μM does not further decrease viability suggesting that PCLX-001 effects are mediated upstream of dasatinib and ibrutinib targets. If PCLX-001 effectively suppresses the initial stages of BCR signaling, the addition of further inhibitors downstream may not yield an additive or synergistic cytotoxic effect (see FIG. 6 and mechanism of action described previously). Essentially, if the signal is already dampened or eliminated at an early stage, additional blockade downstream would provide minimal incremental benefit. The critical reliance of lymphoma cells on the myristoylated proteins might be so profound that once their function is compromised by PCLX-001, the cells may have already initiated a death pathway (e.g., apoptosis or ER stress), leaving little room for ibrutinib or dasatinib to further influence the pathway.

### Example 3: Zelenirstat Combination with Venetoclax or Bendamustine

In this example, the activity of 2 combinations (Venetoclax “ABT-199” vs Zelenirstat and Bendamustine vs Zelenirstat) across a panel of 55 hematological cell lines was tested utilising Horizon Discovery's High Throughput Screening platform. Combinations were dosed in a 9×9 format. Single agents were dosed with 8 dose points including the no treatment control resulting in 64 dose ratio combination points. Principal assay readout was growth inhibition determined using a 96-hour CellTiter-Glo® 2.0 viability assay. Results have been supplied as metrics tables, Chalice Analyzer files and raw luminescence data files.

Venetoclax is a BCL-2 inhibitor that may be used to treat chronic lymphocytic leukemia (CLL)⁴⁰, acute myeloid leukemia (AML), and other hematologic malignancies. It works by selectively binding to BCL-2, an anti-apoptotic protein that prevents programmed cell death in cancer cells. By inhibiting BCL-2, venetoclax restores the apoptotic pathway, allowing cancer cells to undergo programmed cell death. It is particularly effective in tumors that rely on BCL-2 for survival, such as CLL with 17p deletion. Venetoclax may be combined with hypomethylating agents (e.g., azacitidine, decitabine) or rituximab-based regimens to enhance efficacy. One of its main side effects is tumor lysis syndrome (TLS) due to rapid cell death, requiring careful dose escalation and monitoring.

Bendamustine is a chemotherapy agent with both alkylating and antimetabolite properties, that may be used primarily to treat chronic lymphocytic leukemia (CLL), non-Hodgkin's lymphoma (NHL), and multiple myeloma⁴¹. As an alkylating agent, it forms DNA crosslinks, leading to double-strand breaks and apoptosis. Additionally, its purine analog structure disrupts DNA synthesis and repair, enhancing cytotoxicity. Bendamustine is often combined with rituximab (BR regimen) for NHL and CLL, providing an effective alternative to traditional regimens like CHOP. Compared to other alkylating agents, it has a more favorable toxicity profile, with less myelosuppression and lower rates of secondary malignancies.

### Method

Cells were seeded in 25 μl of growth media in black 384-well tissue culture treated plates (Perkin Elmer) at the density displayed in Chalice Analyzer and noted in the cell line table in the associated data file. Cells were equilibrated in assay plates via centrifugation and placed at 37 °C, 5% CO₂ for twenty-four hours before treatment. At the time of treatment, a set of assay plates (which do not receive treatment) were collected and ATP levels quantified by adding CellTiter-Glo® 2.0 (Promega) and luminescence read on Envision plate readers (Perkin Elmer). Compounds were transferred to assay plates using Labcyte Echo acoustic liquid handling systems (Beckman Coulter). Assay plates were incubated with compound for 96 hours and then analysed using CellTiter-Glo® 2.0. All data points were collected via automated processes and are subject to quality control and analysed using Horizon's proprietary software. Horizon utilizes growth inhibition (GI) as a measure of cell growth. The GI percentages are calculated by applying the following test and equation:

If T<V₀:100*(1−(T−V₀)/V₀) If T V₀:100*(1−(T−V₀)/(V−V₀))

where T is the signal measure for a test article, V is the untreated control measure, and V₀ is the untreated measure at time zero (also colloquially referred as TO plates). This formula is derived from the growth inhibition calculation used in the National Cancer Institute's NCI-60 high throughput screen.

A GI reading of 0% represents no growth inhibition and would occur in instances where the T reading is comparable to the V reading at the end of the treatment time. A GI of 100% represents complete growth inhibition (cytostasis) and in this case cells treated for the respective period would have the same endpoint reading as TO control cells. A GI of 200% represents complete death (cytotoxicity) of all cells in the culture well and, in this case, the T reading at the assay endpoint will be lower than the TO control (values near or at zero).

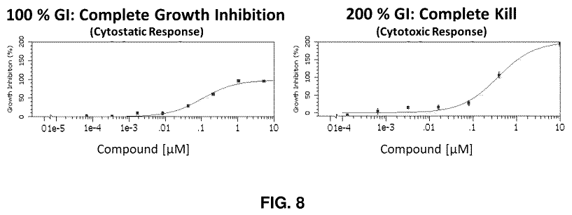

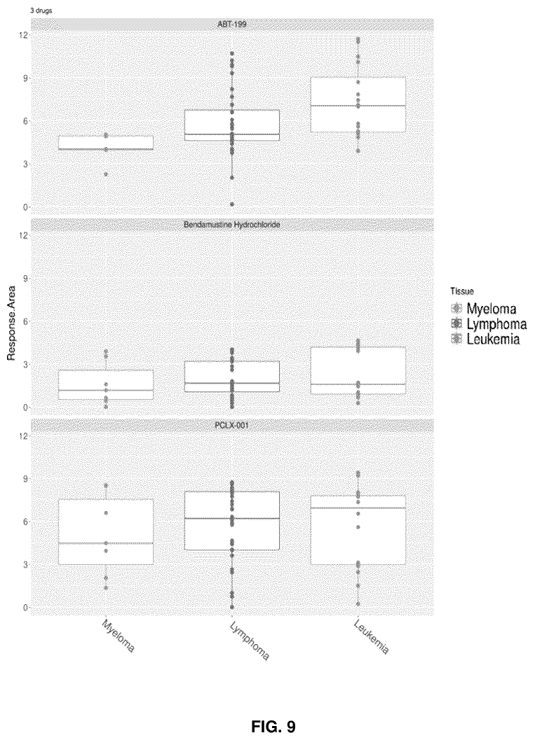

FIG. 8 shows a dose response curve representations of cytostatic vs cytotoxic compound activity.

Horizon also provides inhibition as a measure of cell viability. Inhibition percentage is calculated as following:

I=1−T/U

where T is the treated and U is the untreated/vehicle control.

### Single Agent Curve Fitting

The Horizon Chalice Analyzer software allows two ways of visualising single agent dose response curves. The Logistics Curve fit modeling in Analyzer uses sigmoidal modeling of the data points. In most contexts, the Logistics Curve fit will accurately model the dose response curve. Caution should be used in cases of poor modeling, which can be observed with either particularly steep or shallow dose response curves, or in the case of bi-phasic compound responses. Poor modeling can result in inaccurate reporting of IC/GI values which, in turn, can lead to poor Loewe Additivity combination modeling and artificially high Synergy Scores for combination data.

### Combination Effect Reference Models

Combination effects can be most readily characterised by comparing each data point to that of a combination reference model that was derived from single agent curves (refer to single agent curve fitting section below). Two models are used in this analysis: (1) the Highest Single Agent is a simple reference model where the expected combination effect is the maximum of the single agent responses at corresponding concentrations; and (2) the Loewe Additivity model represents the expected response if both agents are the same compound and is the most generally accepted reference for synergy. HSA is relatively easily calculated, but determining Loewe Additivity is experimentally demanding and requires well-sampled single agent dose curves, data interpolation and iterative root finding for computation of the Loewe additive response surface.

### Highest Single Agent Model

The Highest Single Agent (HSA) model is generally selected for the analysis of combinations where there is a limited dose range or ratio available. Mathematically, the HSA model describes simple superposition of the single agent curves:

I_{HSA}(C_{X},C_{Y})=max(I_{X},I_{Y})

where C_{X,Y} are the concentrations of the X and Y compound, and I_{X} and I_{Y} are inhibitions of the single agents at C_{X,Y}. It is also useful to calculate a volume score (HSA Volume) between the data and the HSA surface to characterise the overall strength of combination effects.

Empirically derived combination matrices are compared to their respective HSA additivity models constructed from experimentally collected single agent dose response curves. Summation of this excess additivity across the dose response matrix is referred to HSA Volume. Positive HSA Volume suggests potential synergy, while negative HSA Volume suggests potential antagonism.

It is important to note that the HSA model is a simple reference model where the expected combination effect is the maximum of the single agent response at corresponding concentrations. Hence, experimental noise of the single agent activity (typically observed below GI 30-50%) can contribute to artificially high HSA Volume Score thereby, adversely affecting the accurate assessment of combination activates. Therefore, upon establishing an HSA Volume threshold, data was manually curated to verify combination interactions observed in the dose matrix.

As other parameters are available for further investigation in the Chalice software, the other combination effect reference models are briefly discussed below.

### Loewe Additivity Model

The Loewe additivity model is dose-based and applies only to the activity levels achieved by the single agents. Loewe Volume is used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for Loewe additivity is:

I_{Loewe} that satisfies(X/X_{I})+(Y/Y_{I})=1

where X_{I} and Y_{I} are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

For the present analysis, as seen in the example below for the combinations of enhancee with enhancer, empirically derived combination matrices were compared to their respective Loewe additivity models constructed from experimentally collected single agent dose response curves. Summation of this excess additivity across the dose response matrix is referred to as Loewe Volume. Positive Loewe volume suggests potential synergy, whilst negative Loewe volume suggests potential antagonism.

### Synergy Score Analysis

To measure combination effects in excess of Loewe additivity, Horizon has devised a scalar measure to characterise the strength of synergistic interaction termed the Synergy Score. The Synergy score is calculated as:

Synergy Score=log f_{X} log f_{Y}Σmax(0,I_{data})(I_{data}−I_{Loewe})

The fractional inhibition for each component agent and combination point in the matrix is calculated relative to the median of all untreated/vehicle-treated control wells. The Synergy Score equation integrates the experimentally-observed activity volume at each point in the matrix in excess of a model surface numerically derived from the activity of the component agents using the Loewe model for additivity. Additional terms in the Synergy Score equation (above) may be used to normalise for various dilution factors used for individual agents and to allow for comparison of synergy scores across an entire experiment. The inclusion of positive inhibition gating or an I_{data} multiplier removes noise near the zero-effect level, and biases results for synergistic interactions at that occur at high activity levels. Combinations with higher maximum Growth Inhibition (GI) effects or those which are synergistic at low concentrations will have higher Synergy Scores.

Potency shifting was evaluated using an isobologram, which demonstrates how much less drug is required in combination to achieve a desired effect level, when compared to the single agent doses needed to reach that effect. The isobologram was drawn by identifying the locus of concentrations that correspond to crossing the indicated inhibition level. This is done by finding the crossing point for each single agent concentration in a dose matrix across the concentrations of the other single agent. Practically, each vertical concentration C_{Y} is held fixed while a bisection algorithm is used to identify the horizontal concentration C_{X} in combination with that vertical dose that gives the chosen effect level in the response surface Z(C_{X},C_{Y}). These concentrations are then connected by linear interpolation to generate the isobologram display. For synergistic interactions, the isobologram contour falls below the additivity threshold and approaches the origin, and an antagonistic interaction would lie above the additivity threshold. The error bars represent the uncertainty arising from the individual data points used to generate the isobologram. The uncertainty for each crossing point is estimated from the response errors using bisection to find the concentrations where Z−σ_{Z}(C_{X},C_{Y}) and Z+σ_{Z}(C_{X},C_{Y}) cross I_{cut}, where σ_{Z} is the standard deviation of the residual error on the effect scale.

### Combination Index

Potency shifting can also be scored using combination index (CI). For a chosen iso-effect level (I_{cut}), CI is calculated as:

CI=(C_{X}/EC_{X})+(C_{Y}/EC_{Y})
where (C_{X}/EC_{X}) for a particular data point is the ratio of the X compound's measured concentration to its effective concentration at the chosen effect level. The CI is a rough estimate of how much drug was needed in combination relative to the single agent doses required to achieve the chosen effect level. CI values in the range of 0.5-0.7 are typical for in vitro measurements of current clinical combinations. The CI error (σCI) is calculated using standard error propagation through the CI calculation based on the isobologram errors. In the Chalice Analyzer, the Best CI is reported from the many CI values calculated for each I_{cut} crossing combination. Among all the measured CI values, the one with the largest signal-to-noise level (1−CI)/σCI is reported as the Best CI. Best CI Level is the effect level from which the CI is calculated.

### Results

### Enhancee Single Agent Activity

### Combination Activity

FIG. 9 depicts box whisker plots showing enhancee single agent response by tissue type. Single Agent activity was extracted from the dose matrices Cell lines are grouped by tissue type (sorted by median response for each tissue type, ABT-199 (venetoclax) used to rank median response area), and agent activity represented by Response Area (area under growth inhibition dose response curve).

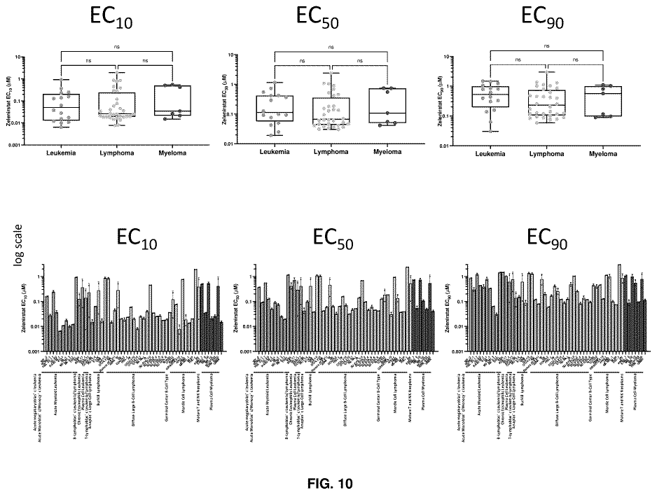

FIG. 10 shows a comparison of the EC₁₀, EC₅₀ and EC₉₀ for the panel of 55 hematological cell lines treated with zelenirstat.

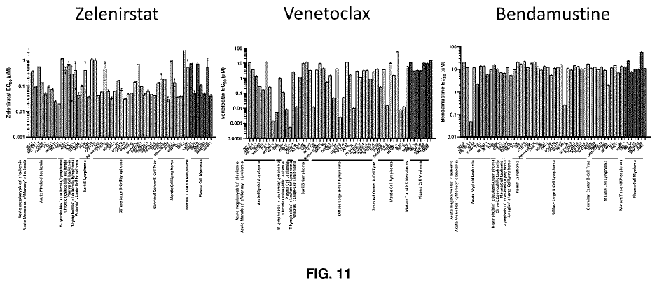

FIG. 11 shows a comparison of the EC₅₀ for the panel of 55 hematological cell lines treated with either zelenirstat, venetoclax or bendamustine.

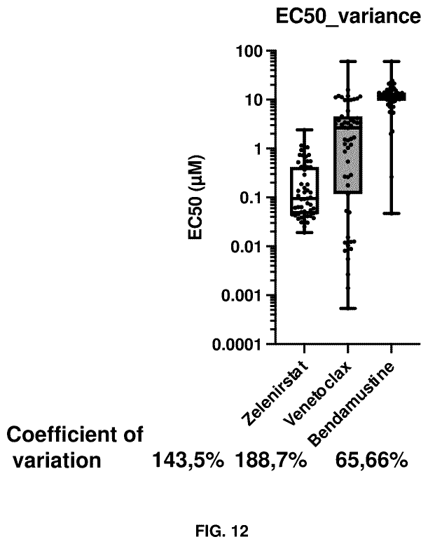

FIG. 12 shows a distribution of EC₅₀ for the panel of 55 hematological cell lines treated with either zelenirstat, venetoclax or bendamustine. EC50 variation for the cells treated with zelenirstat is lower than the EC₅₀ variation for the cells treated venetoclax. Also, hematological cell lines are significantly more sensitive to zelenirstat than bendamustine. Zelenirstat can target efficiently a wider range of hematological cancer cell lines due to its impact on several myristoylated proteins. The coefficient of variation (CV), also known as “relative variability”, equals the standard deviation divided by the mean. It can be expressed either as a fraction or a percent.

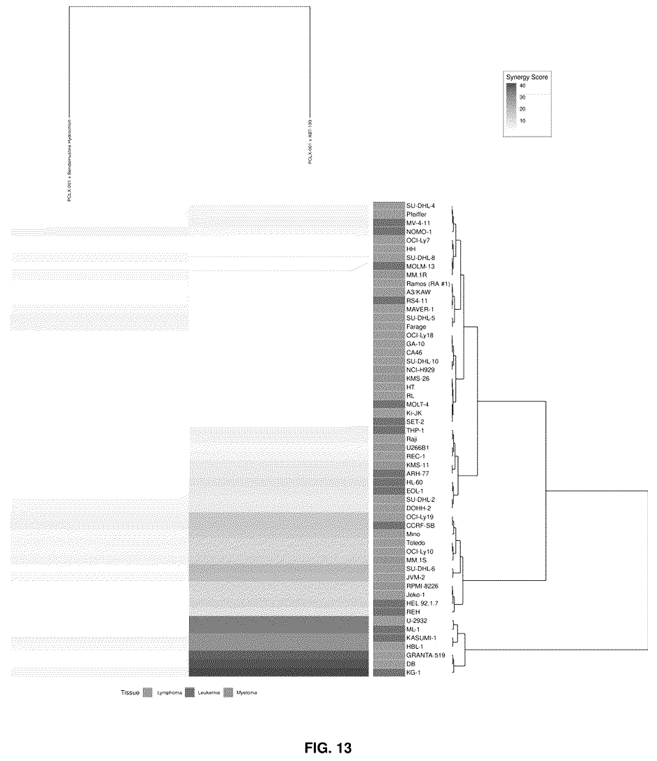

FIG. 13 shows heatmaps of combination activity. Combination activity was represented by Synergy Score as determined by Chalice Analyzer. Cells lines are annotated by tissue indication. Darker red indicates higher synergy. Clustering analysis was performed using the wardD cluster method with the Euclidean distance metric. For the order of combinations and cell lines from cluster analysis, see associated heatmap data file.

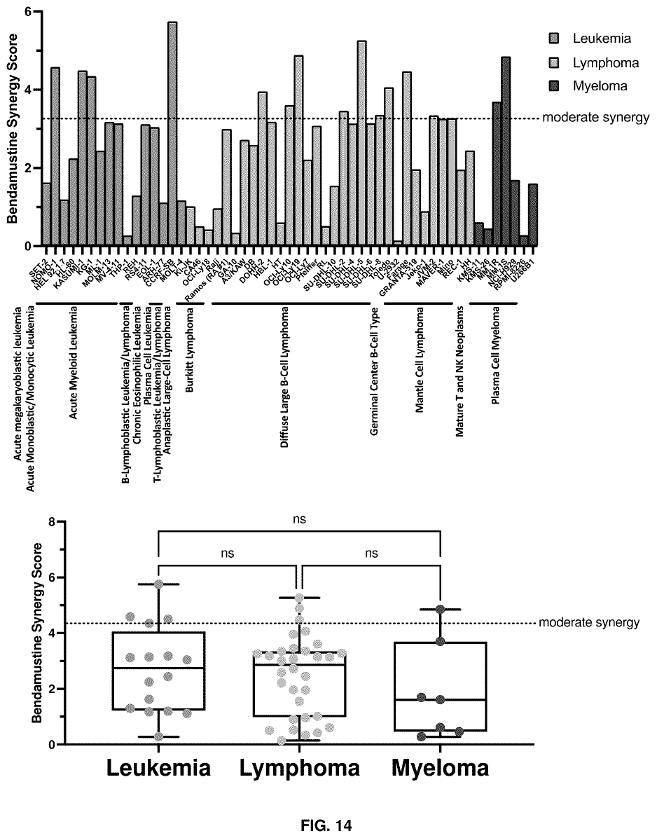

FIG. 14 shows experimental results wherein zelenirstat shows moderate synergy with bendamustine. Hematological cell lines were screened for synergy between zelenirstat and bendamustine using Horizon's Combination Profiling platform. Synergy scores of hematological cell lines in combination with bendamustine. Synergy scores indicate growth inhibition in excess of additive effects. Potential moderate synergistic interactions (scores from 3.25-7.19) and strong synergistic interactions (scores greater than 7.19) are displayed on the histogram.

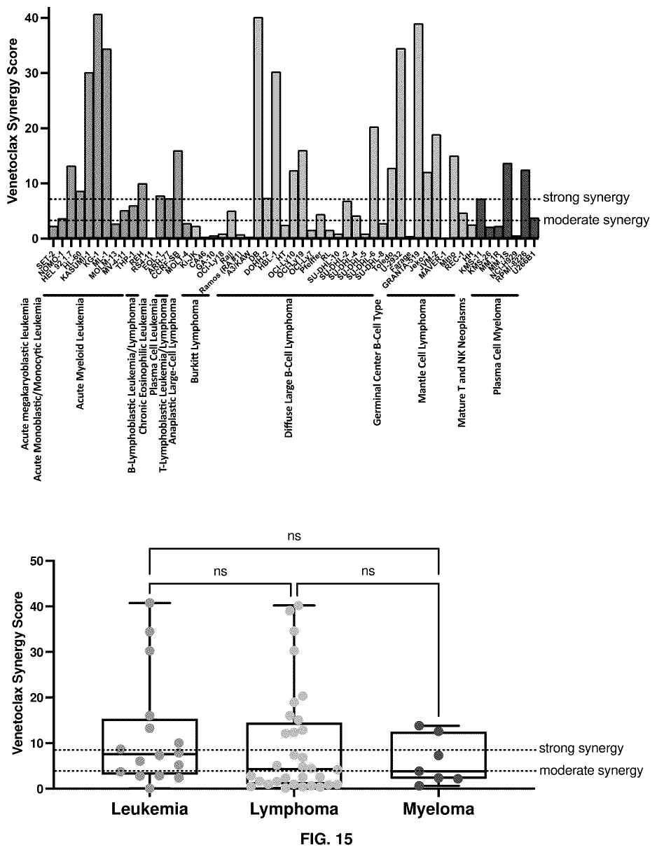

FIG. 15 shows experimental results wherein zelenirstat shows synergy with venetoclax (ABT-199). Hematological cell lines were screened for synergy between zelenirstat and venetoclax using Horizon's Combination Profiling platform. Synergy scores of hematological cell lines in combination with venetoclax. Synergy scores indicate growth inhibition in excess of additive effects. Potential moderate synergistic interactions (scores from 3.25-7.19) and strong synergistic interactions (scores greater than 7.19) are displayed on the histogram.

In this experiment, a panel of 55 hematological cancer cell lines demonstrates that all cells exhibit sensitivity to cytotoxic agents venetoclax (ABT-199), bendamustine, and zelenirstat (FIG. 9). Cancer cells exhibited comparable sensitivity to these three agents when sorted based on cancer indications (Lymphoma, Leukemia, and Myeloma) (FIG. 9). Notably, sensitivity towards zelenirstat was highly consistent across the 55 cell lines panel, even when considering EC₁₀ and EC₉₀ values (FIG. 10). In contrast, this was not the case for venetoclax, where it was evident that certain cell lines exhibited high sensitivity, while others displayed increased resistance (FIGS. 11 and 12). This disparity can be attributed to the highly specific mechanism of action of venetoclax and the cell's reliance on BCL-2 for survival. Furthermore, cells exhibited overall resistance to bendamustine, with an average EC₅₀ value exceeding 12 uM (FIG. 12).

An experiment was conducted to identify potential enhancers and cancer indications suitable for zelenirstat combination therapy, (FIG. 13).

As evident in FIG. 13 heatmap experiment, the therapeutic potential of the combination of zelenirstat and venetoclax was higher to that of zelenirstat and bendamustine.

Notably, only 15 out of 55 cell lines exhibited moderate synergy between bendamustine and zelenirstat, with none demonstrating strong synergy.

In contrast, 60% (33 out of 55) of tested cell lines exhibited at least moderate synergy between zelenirstat and venetoclax, and some cells displayed high synergy (24 out of 55).

Remarkably, this observed synergy was independent of the origin of cancer (leukemia, lymphoma, and myeloma) (FIG. 12). Furthermore, extremely high synergy scores (over 30) were observed in 7 cell lines, indicating a substantial potential for the clinical application of the combination of zelenirstat and venetoclax.

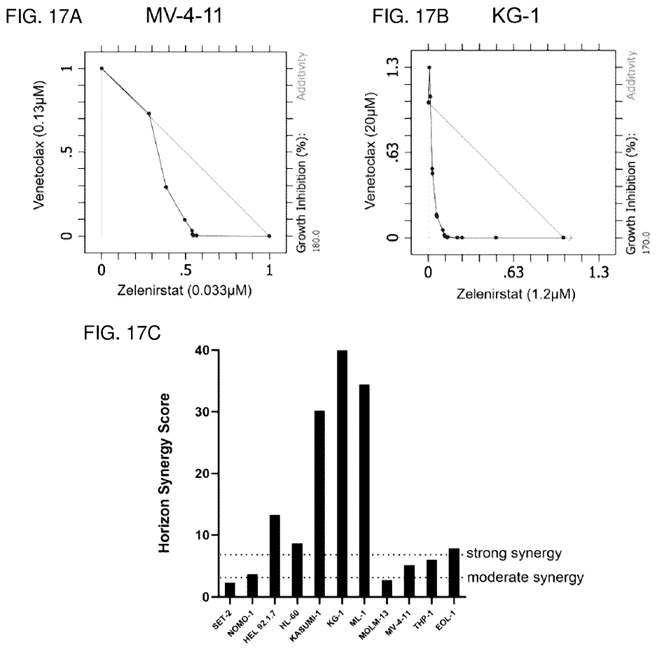

FIG. 17 illustrates Zelenirstat shows synergy with venetoclax. AML cell lines were screened for synergy between zelenirstat and venetoclax using Horizon's Combination Profiling platform. Isobolograms of (A) MV-4-11 and (B) KG-1 cell lines of zelenirstat and venetoclax combination therapy. Trendlines below additivity line indicate synergy. (C) Synergy scores of AML cell lines in combination with venetoclax. Synergy scores indicate growth inhibition in excess of additive effects.

### DISCUSSION

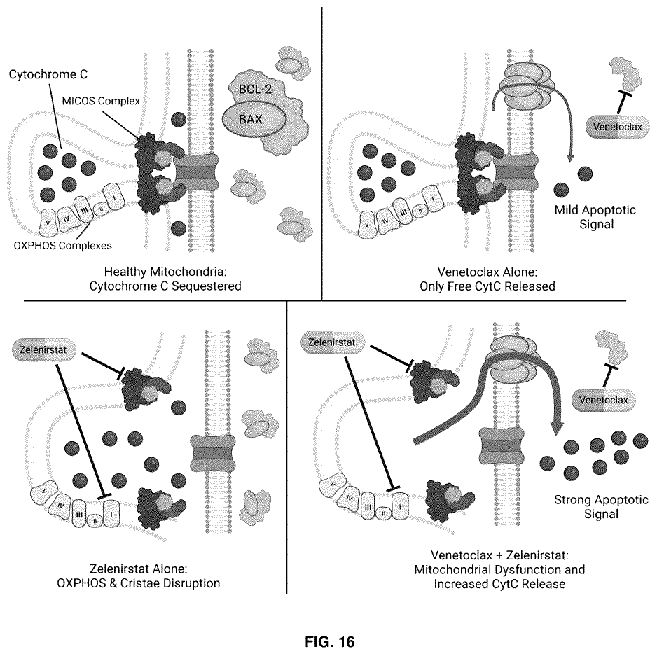

FIG. 16 illustrates a model describing the potential mechanism of action for the strong synergy observed between zelenirstat and venetoclax.

Hypothesis: Zelenirstat may disrupt MICOS/MIB (Mitochondrial contact site/mitochondrial intermembrane space bridging) complex function, compromising cristae structure and liberating cytochrome c for release by venetoclax-mediated BAX/BAK pore formation.

Cytochrome c may be held within cristae, and its release may be tightly regulated by cristae architecture; disrupting the MICOS complex promotes cytochrome c mobilization; and myristoylated Mic19 is vital for cristae integrity-its loss (via cleavage or inhibited myristoylation) may predispose cells to apoptosis. Therefore, Zelenirstat's inhibition of myristoylation may serve as an upstream event that primes mitochondria for venetoclax-mediated apoptosis, suggesting a potential synergistic therapeutic strategy in cancer treatment, as observed in FIG. 15 and hypothesised in FIG. 16.

### CONCLUSION

As described in the present disclosure, combining multiple therapeutically approved drugs with zelenirstat, a novel phase 2 small molecule N-myristoyltransferase inhibitor may be beneficial. Phase 1 study¹¹ demonstrated the safety of administering a myristoylation inhibitor in humans (Maximum Tolerated Dose for Zelenirstat: 210 mg daily). Additionally, early signs of efficacy were observed, with progression-free survival and overall survival significantly improved in patients receiving 210 mg once daily compared to those receiving lower doses.

N-myristoyltransferase inhibitors may primarily target NMT1 and NMT2, but their inhibition may directly affect over 200 myristoylated proteins. The impact of myristoylation inhibition may vary depending on the cancer indication and the pathway it depends on for survival.

### REFERENCES

1. Buss J E, Kamps M P, Sefton B M. Myristic acid is attached to the transforming protein of Rous sarcoma virus during or immediately after synthesis and is present in both soluble and membrane-bound forms of the protein. Mol Cell Biol. 1984; 4(12):2697-2704.
2. Hantschel O, Nagar B, Guettler S, et al. A myristoyl/phosphotyrosine switch regulates c-Abl. Cell. 2003; 112(6):845-857.
3. Yuan M, Song Z H, Ying M D, et al. N-myristoylation: from cell biology to translational medicine. Acta Pharmacol Sin. 2020; 41(8):1005-1015.
4. Hayashi N, Titani K. N-myristoylated proteins, key components in intracellular signal transduction systems enabling rapid and flexible cell responses. Proc Jpn Acad Ser B Phys Biol Sci. 2010; 86(5):494-508.
5. Timms R T, Zhang Z, Rhee D Y, Harper J W, Koren I, Elledge S J. A glycine-specific N-degron pathway mediates the quality control of protein N-myristoylation. Science. 2019; 365(6448).
6. Tate E W, Kalesh K A, Lanyon-Hogg T, Storck E M, Thinon E. Global profiling of protein lipidation using chemical proteomic technologies. Curr Opin Chem Biol. 2015; 24:48-57.
7. Shoji S, Kurosawa T, Inoue H, Funakoshi T, Kubota Y. Human cellular src gene product: identification of the myristoylated pp60c-src and blockage of its myristoyl acylation with N-fatty acyl compounds resulted in the suppression of colony formation. Biochem Biophys Res Commun. 1990; 173(3):894-901.
8. Martin D D, Beauchamp E, Berthiaume L G. Post-translational myristoylation: Fat matters in cellular life and death. Biochimie. 2011; 93(1):18-31.
9. Beauchamp E, Gamma J M, Cromwell C R, et al. Multiomics analysis identifies oxidative phosphorylation as a cancer vulnerability arising from myristoylation inhibition. J Transl Med. 2024; 22(1):431.
10. Brand S, Norcross N R, Thompson S, et al. Lead optimization of a pyrazole sulfonamide series of Trypanosoma brucei N-myristoyltransferase inhibitors: identification and evaluation of CNS penetrant compounds as potential treatments for stage 2 human African trypanosomiasis. J Med Chem. 2014; 57(23):9855-9869.
11. Sangha R, Jamal R, Spratlin J, et al. A first-in-human phase I trial of daily oral zelenirstat, a N-myristoyltransferase inhibitor, in patients with advanced solid tumors and relapsed/refractory B-cell lymphomas. Invest New Drugs. 2024; 42(4):386-393.
12. Kallemeijn W W, Lueg G A, Faronato M, et al. Validation and Invalidation of Chemical Probes for the Human N-myristoyltransferases. Cell Chem Biol. 2019; 26(6):892-900 e894.
13. Beauchamp E, Yap M C, Iyer A, et al. Targeting N-myristoylation for therapy of B-cell lymphomas. Nat Commun. 2020; 11(1):5348.
14. Birsoy K, Wang T, Chen W W, Freinkman E, Abu-Remaileh M, Sabatini D M. An Essential Role of the Mitochondrial Electron Transport Chain in Cell Proliferation Is to Enable Aspartate Synthesis. Cell. 2015; 162(3):540-551.
15. Kahn M. Can we safely target the WNT pathway?Nat Rev Drug Discov. 2014; 13(7):513-532.
16. Mendoza M C, Er E E, Blenis J. The Ras-ERK and PI3K-mTOR pathways: cross-talk and compensation. Trends Biochem Sci. 2011; 36(6):320-328.
17. Engelman J A. Targeting PI3K signalling in cancer: opportunities, challenges and limitations. Nat Rev Cancer. 2009; 9(8):550-562.
18. Huang Y, Goel S, Duda D G, Fukumura D, Jain R K. Vascular normalization as an emerging strategy to enhance cancer immunotherapy. Cancer Res. 2013; 73(10):2943-2948.
19. Kim H J, Yim G W, Nam E J, Kim Y T. Synergistic Effect of COX-2 Inhibitor on Paclitaxel-Induced Apoptosis in the Human Ovarian Cancer Cell Line OVCAR-3. Cancer Res Treat. 2014; 46(1):81-92.
20. E I-Rayes B F, Ali S, Sarkar F H, Philip P A. Cyclooxygenase-2-dependent and -independent effects of celecoxib in pancreatic cancer cell lines. Mol Cancer Ther. 2004; 3(11):1421-1426.
21. Liu B, Yan S, Qu L, Zhu J. Celecoxib enhances anticancer effect of cisplatin and induces anoikis in osteosarcoma via PI3K/Akt pathway. Cancer Cell Int. 2017; 17:1.
22. Zidovetzki R, Levitan 1. Use of cyclodextrins to manipulate plasma membrane cholesterol content: evidence, misconceptions and control strategies. Biochim Biophys Acta. 2007; 1768(6):1311-1324.
23. Fukumura D, Gohongi T, Kadambi A, et al. Predominant role of endothelial nitric oxide synthase in vascular endothelial growth factor-induced angiogenesis and vascular permeability. Proc Natl Acad Sci USA. 2001; 98(5):2604-2609.
24. Hangauer M J, Viswanathan V S, Ryan M J, et al. Drug-tolerant persister cancer cells are vulnerable to GPX4 inhibition. Nature. 2017; 551(7679):247-250.
25. Lappano R, Maggiolini M. GPCRs and cancer. Acta Pharmacol Sin. 2012; 33(3):351-362.
26. Zhu L, Yang K, Ren Z, Yin D, Zhou Y. Metformin as anticancer agent and adjuvant in cancer combination therapy: Current progress and future prospect. Transl Oncol. 2024; 44:101945.
27. Zeh H J, Bahary N, Boone B A, et al. A Randomized Phase II Preoperative Study of Autophagy Inhibition with High-Dose Hydroxychloroquine and Gemcitabine/Nab-Paclitaxel in Pancreatic Cancer Patients. Clin Cancer Res. 2020; 26(13):3126-3134.
28. Degan S, May B L, Jin Y J, et al. Co-Treatment of Chloroquine and Trametinib Inhibits Melanoma Cell Proliferation and Decreases Immune Cell Infiltration. Front Oncol. 2022; 12:782877.
29. Qian W, Wang J, Roginskaya V, et al. Novel combination of mitochondrial division inhibitor 1 (mdivi-1) and platinum agents produces synergistic pro-apoptotic effect in drug resistant tumor cells. Oncotarget. 2014; 5(12):4180-4194.
30. Dai W, Wang G, Chwa J, et al. Mitochondrial division inhibitor (mdivi-1) decreases oxidative metabolism in cancer. Br J Cancer. 2020; 122(9):1288-1297.
31. Yu L, Lu M, Jia D, et al. Modeling the Genetic Regulation of Cancer Metabolism: Interplay between Glycolysis and Oxidative Phosphorylation. Cancer Res. 2017; 77(7):1564-1574.
32. Larsson D E, Wickström M, Hassan S, Oberg K, Granberg D. The cytotoxic agents NSC-95397, brefeldin A, bortezomib and sanguinarine induce apoptosis in neuroendocrine tumors in vitro. Anticancer Res. 2010; 30(1):149-156.
33. Shaik M S, Chatterjee A, Singh M. Effects of monensin liposomes on the cytotoxicity, apoptosis and expression of multidrug resistance genes in doxorubicin-resistant human breast tumour (MCF-7/dox) cell-line. J Pharm Pharmacol. 2004; 56(7):899-907.
34. Toyoshima M, Howie H L, Imakura M, et al. Functional genomics identifies therapeutic targets for MYC-driven cancer. Proc Natl Acad Sci USA. 2012; 109(24):9545-9550.
35. Hart J R, Garner A L, Yu J, et al. Inhibitor of MYC identified in a Kröhnke pyridine library. Proc Natl Acad Sci USA. 2014; 111(34):12556-12561.
36. Lee J V, Housley F, Yau C, et al. Combinatorial immunotherapies overcome MYC-driven immune evasion in triple negative breast cancer. Nat Commun. 2022; 13(1):3671.
37. Ryl T, Kuchen E E, Bell E, et al. Cell-Cycle Position of Single MYC-Driven Cancer Cells Dictates Their Susceptibility to a Chemotherapeutic Drug. Cell Syst. 2017; 5(3):237-250.e238.
38. Thinon E, Morales-Sanfrutos J, Mann D J, Tate E W. N-Myristoyltransferase Inhibition Induces E R-Stress, Cell Cycle Arrest, and Apoptosis in Cancer Cells. ACS Chem Biol. 2016; 11(8):2165-2176.
39. Wang S, Konorev E A, Kotamraju S, Joseph J, Kalivendi S, Kalyanaraman B. Doxorubicin induces apoptosis in normal and tumor cells via distinctly different mechanisms. intermediacy of H(2)O(2)— and p53-dependent pathways. J Biol Chem. 2004; 279(24):25535-25543.
40. Molica S. Venetoclax: a real game changer in treatment of chronic lymphocytic leukemia. Int J Hematol Oncol. 2020; 9(4):IJH31.
41. Cheson B D, Brugger W, Damaj G, et al. Optimal use of bendamustine in hematologic disorders: Treatment recommendations from an international consensus panel—an update. Leuk Lymphoma. 2016; 57(4):766-782.
42. Heath-Engel H M, Shore G C. Mitochondrial membrane dynamics, cristae remodelling and apoptosis. Biochim Biophys Acta. 2006; 1763(5-6):549-560.
43. Scorrano L, Ashiya M, Buttle K, et al. A distinct pathway remodels mitochondrial cristae and mobilizes cytochrome c during apoptosis. Dev Cell. 2002; 2(1):55-67.
44. Lin C Y, Huang Y C, Wu C R, Wu H T, Fu R H, Tsai C W. Carnosic acid attenuated cytochrome c release through the mitochondrial structural protein Mic60 by PINK1 in SH-SY5Y cells. Food Chem Toxicol. 2023; 173:113636.
45. Bock-Bierbaum T, Funck K, Wollweber F, et al. Structural insights into crista junction formation by the Mic60-Mic19 complex. Sci Adv. 2022; 8(35):eabo4946.
46. Tang J, Zhang K, Dong J, et al. Sam50-Mic19-Mic60 axis determines mitochondrial cristae architecture by mediating mitochondrial outer and inner membrane contact. Cell Death Differ. 2020; 27(1):146-160.
47. Yang S, Yin X, Wang J, et al. MIC19 Exerts Neuroprotective Role via Maintaining the Mitochondrial Structure in a Rat Model of Intracerebral Hemorrhage. Int J Mol Sci. 2023; 24(14).
48. Gamma J M, Liu Q, Beauchamp E, et al. Zelenirstat Inhibits N-Myristoyltransferases to Disrupt Src Family Kinase Signalling and Oxidative Phosphorylation Killing Acute Myeloid Leukemia Cells. Mol Cancer Ther. 2024.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A method of treating cancer in a subject comprising: administering a therapeutically effective amount of PCLX-001 and a therapeutically effective amount of cytarabine, wherein the subject is a human.

2. The method of claim 1, wherein the cancer is bladder cancer, breast cancer, colorectal cancer, leukemia, lung cancer, lymphoma, myeloma, pancreatic cancer or skin cancer.

3. A method of treating bladder cancer, breast cancer, colorectal cancer, leukemia, lung cancer, lymphoma, myeloma, pancreatic cancer or skin cancer in a subject comprising:
administering a therapeutically effective amount of PCLX-001 and a therapeutically effective amount of doxorubicin, wherein the subject is a human.

4. The method of claim 3, wherein the cancer is bladder cancer, breast cancer, colorectal cancer, leukemia, lung cancer, lymphoma, myeloma, pancreatic cancer or skin cancer.

5. A method of treating cancer in a subject comprising: administering a therapeutically effective amount of PCLX-001 and a therapeutically effective amount of venetoclax or bendamustine, wherein the subject is a human.

6. The method of claim 5, wherein the cancer is a hematologic cancer.

7. The method of claim 6, wherein the hematologic cancer is myeloma, lymphoma, or leukemia.

8. The method of claim 7, wherein the myeloma is plasma cell myeloma.

9. The method of claim 7, wherein the lymphoma is B-lymphoblastic lymphoma, T-lymphoblastic lymphoma, anaplastic large-cell lymphoma, Burkitt lymphoma, diffuse large B-cell lymphoma, germinal center B-cell type lymphoma, mantle cell lymphoma, or mature T and natural killer neoplasm.

10. The method of claim 7, wherein the leukemia is acute megakaryoblastic leukemia, acute monoblastic/monocytic leukemia, acute myeloid leukemia, B-lymphoblastic leukemia, chronic eosinophilic leukemia, plasma cell leukemia, or T-lymphoblastic leukemia.
